(19) United States
(12) Patent Application Publication
(10) Pub. No.: US 2017/0242269 A1
(43) Pub. Date: Aug. 24, 2017

(54) CONTACT LENSES AND METHODS OF MAKING CONTACT LENSES

(71) Applicants: Karen L. HAVENSTRITE, San Francisco, CA (US); Victor Wayne MCCRAY, San Jose, CA (US); Brandon McNary FELKINS, Half Moon Bay, CA (US); Andrew A. MCGIBBON, San Francisco, CA (US)

(72) Inventors: Karen L. HAVENSTRITE, San Francisco, CA (US); Victor Wayne MCCRAY, San Jose, CA (US); Brandon McNary FELKINS, Half Moon Bay, CA (US); Andrew A. MCGIBBON, San Francisco, CA (US)

(21) Appl. No.: 15/591,872

(22) Filed: May 10, 2017

Related U.S. Application Data
(63) Continuation of application No. 15/325,678, filed on Jan. 11, 2017.
(60) Provisional application No. 62/027,177, filed on Jul. 21, 2014.

(51) Int. Cl. G02C 7/04; A61F 9/00; B29D 11/00

(52) U.S. Cl. CPC G02C 7/049; B29D 11/00865; A61F 9/0017

(57) ABSTRACT

Contact lenses with hydrophilic polymer coatings are described herein along with methods of making such lenses. The contact lenses can include a lens core that comprises about 75% to about 100% silicone. The hydrophilic polymer coating can include polyethylene glycol and polyacrylamide.

### CROSS REFERENCE TO RELATED APPLICATIONS

This application is a continuation of U.S. patent application Ser. No. 15/325,678, filed Jan. 11, 2017, which is a national stage filing under 35 U.S.C. §371 of PCT/US15/41119, filed Jul. 20, 2015, which claims priority to U.S. Provisional Application Ser. No. 62/027,177 filed on Jul. 21, 2014, each of which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

Embodiments of the technology relate to a soft contact lens with improved oxygen permeability, biocompatibility, wettability, lubricity and wearability and methods for making the improved lens. More particularly, the technology relates to a contact lens with a high oxygen permeable core and a highly stable, hydrophilic, bio-inspired coating layer comprising a polymer and/or polysaccharide analogue to improve surface performance.

### BACKGROUND

Contact lenses are medical devices that are placed in contact with the ocular surface and are used for vision correction, aesthetic purposes, and to treat ocular pathologies. Substances and materials can be deposited onto a contact lens's surface to improve the biocompatibility of the lens and therefore improve the interaction of the lens with the ocular region.

The current generation of contact lenses commonly includes a silicone containing core material. Silicone containing lenses have the advantage of improved oxygen permeability, which aids in maintaining normal ocular surface health. However, a major challenge for silicone containing lenses is the hydrophobicity of silicone containing materials, which can cause poor interaction between the contact lens and the ocular surface resulting in disruption of the tear film and ocular discomfort. The problem of hydrophobicity has been ameliorated in several lens designs by the addition of a water based hydrogel polymer component to the contact lens, thereby improving its hydrophilicity. These combined silicone and hydrogel designs have been termed silicone-hydrogels, and are now the dominant lens type in the industry. Although the addition of water to the core lens improves the hydrophilicity, this also decreases its oxygen permeability. Therefore, a delicate balance exists compromising corneal health with wearing comfort. Plasma surface treatments have been used to improve the hydrophilicity of soft lens surfaces, however these thin layers do little to mask the underlying lens material, and therefore the core lenses still require a relatively high water content to allow comfortable wear. As such, embodiments described herein provide for a contact lens having high oxygen permeability in addition to improved hydrophilicity and biocompatibility as well as practical and cost-effective methods for making these lenses.

An additional challenge with contact lens technology is the tendency for protein binding and absorption at the ocular site. For example, a contact lens may bind proteins on the lens to create protein deposits in the eye area. Additionally, the lens can cause structural changes including protein denaturation that can elicit an immune response such as tearing, reddening, or swelling in the ocular region. Accordingly, contemplated embodiments provide for contact lenses and methods of making lenses with improved resistance to undesirable protein interactions at the ocular site.

A further concern with contact lens use is that some users experience discomfort that is similar to the profile of patients that have a dry eye disease. Dry eye disease is considered to be a consequence of a disruption of the tear film that covers the surface of the eye, or a particular vulnerability to such disruption. This tear film is an aqueous layer disposed between an underlying mucous layer that is secreted by corneal cells, and an overlying lipid layer that is secreted by Meibomian glands on the conjunctival surface of the eyelids. The mucin layer consists of protein tethered to the cornea and integrated polysaccharides with an affinity for the aqueous tears. The tear film includes an aqueous pool that transits across the eye surface, having a flow path that, to some degree, may be independent of the lipid layers that it is disposed between at any point in time. This aqueous pool complexes with the mucin/polysaccharides to create a moisture layer on the corneal surface. Accordingly, contemplated embodiments provide for contact lenses and methods of making lenses with polysaccharides or analogues to improve the lenses' affinity for tears.

Integrity of the tear film is important for such critical functions as oxygen and ion transport, and lubricating the eye surface, which is subject to a constant sliding contact by the eyelids. It is likely that dry eye disease actually exists as a spectrum of tear film vulnerability to disruption. In some cases, patients may have a low-level dry eye disease that manifests when the integrity of the film is challenged by the presence of a contact lens. To address this concern, some embodiments of the invention provide for contact lens technology that diminishes or substantially eliminates contact lens disruption of the tear film.

As can be appreciated, dry eye disease may be referred to herein as a non-limiting example for illustration purposes. The methods and devices described may be used to treat or prevent other ocular pathologies including, but not limited to, glaucoma, corneal ulcers, scleritis, keratitis, iritis, and corneal neovascularization.

### SUMMARY OF THE DISCLOSURE

Some embodiments of the invention provide for a highly oxygen permeable, polymer coated soft contact lens including a silicone containing lens core comprising an outer surface and a hydrophilic, polymer coating layer covalently attached to at least a portion of the outer surface, the coating layer adapted to contact an ophthalmic surface, wherein the coating layer comprises a hydrophilic polymer population having a first polymer species and a second polymer species, the first polymer species being at least partially cross-linked to the second polymer species.

In any of the preceding embodiments, the coating layer comprises a polysaccharide that is at least partially cross-linked to the hydrophilic polymer population.

In any of the preceding embodiments, the coating layer comprises a pharmaceutical.

In any of the preceding embodiments, the contact lens is a silicone contact lens. In any of the preceding embodiments, the contact lens has a soft silicone core. In any of the preceding embodiments, the soft silicone core comprises silicone.

In any of the preceding embodiments, the contact lens is a silicone-hydrogel contact lens. In any of the preceding embodiments, the contact lens has a silicone-hydrogel core. In any of the preceding embodiments, the silicone-hydrogel core comprises silicone. In any of the preceding embodiments, the lens core layer comprises a silicone-hydrogel lens material.

In any of the preceding embodiments, the contact lens core may be cast molded. In any of the preceding embodiments, the contact lens core may be lathe cut. In any of the preceding embodiments, the contact lens core may be injection molded. In any of the preceding embodiments, the contact lens core may be partially cast molded and partially lathe cut.

In any of the preceding embodiments, the oxygen permeability of the contact lens has a Dk between 150 and 500*10̂-11 (cm/sec)(ml O2/ml×mm Hg). In any of the preceding embodiments, the oxygen permeability has a Dk between 250 and 400. In any of the preceding embodiments, the oxygen permeability has a Dk greater than 200.

In any of the preceding embodiments, the coating layer substantially surrounds the outer surface of the core.

In any of the preceding embodiments, the coating layer and core are substantially optically clear. In any of the preceding embodiments, the hydrophilic coating layer is adapted to allow optical transmission through the hydrophilic coating layer to the ophthalmic surface.

In any of the preceding embodiments, the hydrophilic coating layer comprises a thickness between about 1 nm to about 500 nm. In any of the preceding embodiments, the hydrophilic coating layer comprises a thickness between about 1 nm to about 50 nm. In any of the preceding embodiments, the hydrophilic coating layer comprises a thickness between about 10 nm to about 30 nm. In any of the preceding embodiments, the hydrophilic coating layer comprises a thickness below about 100 nm. In any of the preceding embodiments, the hydrophilic coating layer comprises a thickness below about 50 nm. In any of the preceding embodiments, the hydrophilic coating layer comprises a thickness below about 40 nm. In any of the preceding embodiments, the hydrophilic coating layer comprises a maximum thickness of about 10 microns.

In any of the preceding embodiments, a first portion of the hydrophilic coating layer comprises a first thickness different from a second thickness of a second portion of the hydrophilic coating layer.

In any of the preceding embodiments, each of the first and second polymer species is a branched species having a branch count between two to twelve branch arms.

In any of the preceding embodiments, the first polymer species comprises a reactive electron pair accepting group and the second polymer species comprises a reactive nucleophilic group, the reactive electron pair accepting group and the reactive nucleophilic group adapted to react to thereby form cross-links between the first polymer species to the second polymer species. In any of the preceding embodiments, the reactive electron pair accepting group is a sulfone moiety. In any of the preceding embodiments, the reactive nucleophlic group is a thiol moiety.

In any of the preceding embodiments, the reactive electron pair accepting group of the first polymer species is covalently linked to the outer surface of the core.

In any of the preceding embodiments, the coated lens includes an advancing contact angle between about 20 degrees to about 60 degrees. In some embodiments, the advancing contact angle is between about 30 degrees to about 55 degrees.

In any of the preceding embodiments, the hydrophilic polymer layer comprises one or more species of a polymer.

In any of the preceding embodiments, the hydrophilic polymer layer comprises one or more species of a branched polymer. In any of the preceding embodiments, the polymer species comprises a branch count between about two arms to about twelve arms. In any of the preceding embodiments, the branched polymer polymer species comprises starred branching.

In any of the preceding embodiments, the hydrophilic polymer layer is comprised of a polymer selected from a group consisting of polyethylene glycol, or polyacrylamide.

In any of the preceding embodiments, each of the first and second polymer macromers has a molecular weight between about 1 kDa and about 40 kDa. In any of the preceding embodiments, the molecular weight is between about 5 kDa and about 30 kDa.

In any of the preceding embodiments, the hydrophilic polymer layer comprises between about 70% and about 98% water by weight. In any of the preceding embodiments, the hydrophilic polymer layer comprises between about 80% and about 95% water by weight.

In any of the preceding embodiments, the hydrophilic polymer layer comprises at least one polysaccharide. In any of the preceding embodiments, at least one of the polysaccharides is selected from the group consisting of sulfated or non-sulfated polysaccharides. In any of the preceding embodiments, at least one of the polysaccharides is selected from the group consisting of dextran, dextran sulfate, hydroxymethyl propylcellulose, chondrointin, chondrointin sulfate, alginic acid derivatives, heparin, heparin sulfate, hyaluronic acid, cellulose, agarose, chitin, pectin, carrageenan or xylan.

In any of the preceding embodiments, the hydrophilic polymer layer comprises at least one polysaccharide analogue. In any of the preceding embodiments, the polysaccharide analogue may comprise a sulfated, branched polymer.

In any of the preceding embodiments, the hydrophilic polymer layer comprises at least one glycosylated protein. In any of the preceding embodiments, at least one of the proteins comprises mucin.

In any of the preceding embodiments, the hydrophilic polymer layer further comprises at least one active agent. In any of the preceding embodiments, the at least one active agent is selected from the group consisting of a UV-absorbing agent, a visibility tinting agent, an antimicrobial agent, a bioactive agent, a leachable lubricant, a leachable tear-stabilizing agent, or any mixture thereof.

Another aspect of the invention relates to a method of making a hydrophilic polymer coated contact lens including the steps of reacting an outer surface of the contact lens with a first polymer species of a hydrophilic polymer solution, wherein the first polymer species comprises an electron pair accepting moiety and a first portion of the electron pair accepting moiety forms a covalent attachment to the outer surface of the contact lens through a first nucleophlic conjugate reaction; and reacting the first polymer species of the hydrophilic polymer solution with a second polymer species of the hydrophilic polymer solution, the second polymer species comprising a nucleophilic reactive moiety adapted to covalently link to a second portion of the electron pair accepting moiety of the first polymer species in a second nucleophilic conjugate reaction to thereby at least partially cross-link the first and second polymer species, wherein a polymer hydrogel coating is formed and covalently attached to the outer surface of the contact lens by the first and second nucleophilic conjugate reactions.

In any of the preceding embodiments, further including the step of modifying an outer surface of a contact lens to form the plurality of reactive nucleophilic sites on the outer surface. In any of the preceding embodiments, the modifying step comprises exposing the outer surface of the contact lens to a gas plasma treatment.

In any of the preceding embodiments, further including the step of modifying an outer surface of a contact lens to form the plurality of reactive nucleophilic sites on the outer surface. In any of the preceding embodiments, the modifying step comprises adding a chemical activator to the contact lens monomer mix.

In any of the preceding embodiments, the step of reacting an outer surface of the contact lens with the first polymer species includes reacting at least a portion of the plurality of reactive nucleophilic sites on the outer surface with the first portion of the electron pair accepting moiety on the first polymer species.

In any of the preceding embodiments, both of the first and second nucleophilic conjugate reactions are 1,4-nucleophilic addition reactions.

In any of the preceding embodiments, the first and second nucleophilic conjugate reactions are both a Michael-type reaction.

In any of the preceding embodiments, both of the first and second nucleophilic conjugate reactions are click reactions.

In any of the preceding embodiments, the nucleophilic reactive moiety of the second polymer species is a thiol group and the electron pair accepting moiety of the first polymer species is a sulfone group.

In any of the preceding embodiments, the first polymer species and the second polymer species are cross-linked through a thioether moiety.

In any of the preceding embodiments, the hydrophilic polymer solution comprises substantially equivalent concentrations of the first and second polymer species.

In any of the preceding embodiments, the hydrophilic polymer solution comprises the first and second polymer species and a polysaccharide or polysaccharide analogue.

In any of the preceding embodiments, the hydrophilic polymer solution comprises the first polymer species and a polysaccharide or polysaccharide analogue.

In any of the preceding embodiments, the concentration of the electron pair accepting moiety of the first polymer species exceeds the concentration of the nucleophilic reactive moiety of the second polymer species by about 1% to about 30%. In any of the preceding embodiments, the concentration of the electron pair accepting moiety of the first polymer species exceeds the concentration of the nucleophilic polymer reactive moiety of the second polymer species by about 5% and about 20%.

In any of the preceding embodiments, the reacting steps are performed at a temperature between about 15 degrees Celsius and about 150 degrees Celsius. In any of the preceding embodiments, the reacting steps are performed at a temperature between about 20 degrees Celsius and about 60 degrees Celsius. In any of the preceding embodiments, the reacting steps are performed at a temperature between about 100 degrees Celsius and about 150 degrees Celsius.

In any of the preceding embodiments, the reacting steps are performed at a pH between about 5 and about 11. In any of the preceding embodiments, the reacting steps are performed at a pH between about 6 and about 9. In any of the preceding embodiments, the reacting steps are performed at a pH between about 7 and about 9.

In an exemplary embodiment, the invention is a contact lens comprising: a silicone comprising contact lens core and a first hydrophilic polymer layer; wherein said contact lens has a layered structural configuration; the subunits of the polymer of said first hydrophilic polymer layer are comprised of polyethylene glycol and sulfated polyacrylamide subunits; and the first hydrophilic polymer layer and the silicone elastomer contact lens core are covalently attached.

In another embodiment, according to the above paragraph, further comprising a second hydrophilic polymer layer; wherein the subunits of the polymer of said second hydrophilic polymer layer are comprised of polyethylene glycol and sulfated polyacrylamide subunits; and the second hydrophilic polymer layer and the silicone comprising contact lens core are covalently attached.

In an exemplary embodiment, according to any of the above paragraphs, said contact lens comprises an anterior surface and a posterior surface, and wherein said layered structural configuration of the anterior surface is the first hydrophilic polymer layer and the posterior surface is the contact lens core, or the anterior surface is the contact lens core and the posterior surface is the first hydrophilic polymer layer.

In an exemplary embodiment, according to any of the above paragraphs, said contact lens comprises an anterior surface and a posterior surface, and wherein said layered structural configuration is the anterior surface is the first hydrophilic polymer layer and the posterior surface is the second hydrophilic polymer layer.

In an exemplary embodiment, according to any of the above paragraphs, the invention further comprises an inner layer, wherein said contact les core is said inner layer.

In an exemplary embodiment, according to any of the above paragraphs, said contact lens has a contact angle of between about 20 degrees and about 55 degrees.

In an exemplary embodiment, according to any of the above paragraphs, said first hydrophilic polymer layer is essentially non-swellable.

In an exemplary embodiment, according to any of the above paragraphs, said first hydrophilic polymer layer is essentially non-swellable and said second hydrophilic polymer layer is essentially non-swellable.

In an exemplary embodiment, according to any of the above paragraphs, the core lens is substantially uniform in thickness, and the first hydrophilic polymer is substantially uniform in thickness.

In an exemplary embodiment, according to any of the above paragraphs, the second hydrophilic polymer layer is substantially uniform in thickness, and the anterior and posterior hydrophilic polymer layers merge at the peripheral edge of the contact lens to completely enclose the silicone-containing layer.

In an exemplary embodiment, according to any of the above paragraphs, the core lens has an average thickness of between about 10 micron and about 50 microns.

In an exemplary embodiment, according to any of the above paragraphs, the core lens has an average thickness of between about 50 microns and about 100 microns.

In an exemplary embodiment, according to any of the above paragraphs, the core lens has an average thickness of between about 100 microns and about 250 microns.

In some embodiments, according to any of the above paragraphs, the first hydrophilic polymer layer has an average thickness of between about 10 nm and about 50 nm. In some embodiments the first hydrophilic polymer layer has an average thickness of less than about 50 nm or less than about 40 nm.

In some embodiments, according to any of the above paragraphs, the second hydrophilic polymer layer has an average thickness of between about 10 nm and about 50 nm. In some embodiments the second hydrophilic polymer layer has an average thickness of less than about 50 nm or less than about 40 nm.

In general, in one embodiment, a contact lens including a contact lens core comprising about 75% to about 100% silicone and; a coating layer covalently attached to at least a portion of the outer surface, the coating layer adapted to contact an ophthalmic surface, wherein the coating layer comprises a crossed linked, hydrophilic polymer, wherein the contact lens has an oxygen permeability Dk greater than 200 *10̂-11 (cm/sec)(ml 02/ml×mm Hg).

This and other embodiments can include one or more of the following features. The contact lens core can include 50% to 100% silicone. The contact lens core can include 75% to 100% silicone. The contact lens core can include 98% to 100% silicone. The contact lens core can consist of silicone. The contact lens can have an oxygen permeability Dk greater than 200 *10̂-11 (cm/sec)(ml O2/ml×mm Hg). The contact lens can have an oxygen permeability Dk greater than 250 *10̂-11 (cm/sec)(ml O2/ml×mm Hg). The contact lens can have an oxygen permeability Dk greater than 300 *10̂-11 (cm/sec)(ml O2/ml×mm Hg). The contact lens surface can have an advancing contact angle <65 degrees. The contact lens surface can have an advancing contact angle <60 degrees. The contact lens surface can have an advancing contact angle between <55 degrees. The contact lens surface can have an advancing contact angle <50 degrees. The contact lens surface can have an advancing contact angle <45 degrees. The contact lens surface can have has an advancing contact angle <40 degrees. The contact lens surface can have an advancing contact angle <35 degrees. The contact lens surface can have an advancing contact angle <30 degrees. The coating layer and core can be covalently attached at the outer surface by an amine moiety. The coating layer and core can be covalently attached at the outer surface by an epoxide moiety. The first polymer species can include a reactive sulfonyl group and the second polymer species can include a reactive thiol, and the first polymer species and second polymer species can be cross-linked by a thioether linkage. The first polymer species can include a reactive sulfonyl group and the second polymer species can include a reactive amine, and the first polymer species and second polymer species can be cross-linked by a aminoether linkage. The coating layer can substantially surround the outer surface of the core. The coating layer and core can be substantially optically clear. The coating layer can be adapted to allow optical transmission through the coating layer to the ophthalmic surface. The coating layer can include a thickness between about 5 nm to about 30 nm. The coating layer can include a thickness between about 10 nm to about 50 nm. The coating layer can include a maximum thickness of about 10 microns. A first portion of the coating layer can include a first thickness different from a second thickness of a second portion of the coating layer. Each of the polymer species can be a branched species and can have a branch count between two to twelve branch arms. The polymer species can include a reactive electron pair accepting group and the polysaccharide species can include a reactive nucleophilic group, the reactive electron pair accepting group and the reactive nucleophilic group can be adapted to react to thereby form cross-links between the polymer species to the polysaccharide species. The reactive electron pair accepting group can be a sulfonyl moiety. The reactive nucleophilic group can be a amine moiety. The reactive electron pair accepting group of the polysaccharide species can be covalently linked to the outer surface of the core. The coating layer can include between about 80% to about 98% water by weight. The polymer can include polyethylene glycol. The polymer can include polyacrylamide. The polysaccharide can include Chondroitin. The polysaccharide can include Chondroitin sulfate. The polysaccharide can include Dextran. The polysaccharide can include Dextran sulfate. The polysaccharide can include Hydroxyl propyl methyl cellulose.

In general, in one embodiment, a method of making the contact lens includes reacting an outer surface of the contact lens with a first polymer species of a hydrophilic polymer solution, wherein the first polymer species includes an electron pair accepting moiety and a first portion of the electron pair accepting moiety forms a covalent attachment to the outer surface of the contact lens through a first nucleophilic conjugate reaction; and reacting the first polymer species of the hydrophilic polymer solution with a second polymer species of the hydrophilic polymer solution, the second polymer species including a nucleophilic reactive moiety adapted to covalently link to a second portion of the electron pair accepting moiety of the first polymer species in a second nucleophilic conjugate reaction to thereby at least partially cross-link the first and second polymer species, wherein a polymer hydrogel coating is formed and covalently attached to the outer surface of the contact lens by the first and second nucleophilic conjugate reactions.

This and other embodiments can include one or more of the following features. The method can further include modifying an outer surface of a contact lens to form a plurality of chemically reactive nucleophilic sites on the outer surface. The method can further include modifying an outer surface of a contact lens to form a plurality of moieties that physically attract the polymer species to the lens surface. The method can further include modifying an outer surface of a contact lens to form a combination of a plurality of chemically reactive sites as well as a plurality of physically attractive sites on the outer surface. The modification can include exposing the outer surface of the contact lens to a gas plasma treatment. The reactive nucleophilic sites on the outer surface can include amines. The moieties on the outer surface can include carboxylic acids. The modification can include the addition of an activator to the core lens chemical mix. The activator can participate in the radical polymerization process of the core lens during fabrication. The activator can be a bifunctional polyethylene glycol. At least one moiety of the bifunctional activator may not participate in the radical polymerization process of the core lens during fabrication. The activator can covalently bond to the silane backbone of the core lens. The activator can be N-(3-Aminopropyl)methacrylamide hydrochloride. Reacting an outer surface of the contact lens with the first polymer species can include reacting at least a portion of the plurality of reactive nucleophilic sites on the outer surface with the first portion of the electron pair accepting moiety on the first polymer species. The nucleophilic conjugate reactions can be 1,4-nucleophilic addition reactions. The nucleophilic conjugate reactions can be Michael-type reactions. The nucleophilic conjugate reactions can be click reactions. The nucleophilic reactive moiety of the second polymer species can be a thiol group and the electron pair accepting moiety of the first polymer species can be a sulfonyl group. The first polymer species and the second polymer species can be cross-linked through an aminoether moiety. The nucleophilic reactive moiety of the second polymer species can be an amine group and the electron pair accepting moiety of the first polymer species can be a sulfonyl group. The first polymer species and the second polymer species can be cross-linked through a aminoether moiety. The nucleophilic reactive moiety of the second polymer species can be an amine group and the electron pair accepting moiety of the polysaccharide species can be a sulfonyl group.

The first polymer species and the polysaccharide species can cross-linked through an aminoether moiety. The hydrophilic polymer solution can include substantially equivalent concentrations of the reactive moieties of the first polymer species and second polymer species. The concentrations of the reactive moieties of the first polymer species can exceed the concentration of the nucleophilic reactive moiety of the second polymer species by about 1% to about 50%. The reacting steps can be performed at a temperature between about 15 degrees Celsius and about 60 degrees Celsius. The reacting steps can be performed at a temperature of 120 degrees Celsius and 17 barr pressure. The reacting steps can be performed at a pH between about 7 and about 12. The hydrophilic polymer coating can be substantially optically clear. The contact lens can include a core consisting of silicone. The contact lens can include a core comprising silicone.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows a contact lens having a concave and convex surfaces.

FIG. 1B is a cross-sectional view of an exemplary contact lens with a covalently attached cross-linked hydrogel layer.

FIG. 2 is a cross-sectional view of the contact lens shown in FIG. 1B on the cornea.

FIGS. 3A-3B show a first polymer species and a second polymer species with respective reactive groups A and N.

FIGS. 4A-4B show a reaction between a sulfonyl and thiol group.

FIGS. 5A-5C show schematically a hydrophilic polymer having two species covalently attached to a lens core.

FIGS. 6A-6C show a captive bubble test.

FIG. 7 shows an activated lens surface.

FIG. 8 is a schematic diagram of a first and second reaction with principal reactants.

FIGS. 9A-9D show more details of reactants and reactions depicted in FIG. 8.

FIGS. 10A-10B are flow diagrams of exemplary methods described.

FIGS. 11A-11B show a schematic viewing of a continuously stirred tank reactor.

FIGS. 12A-12B show a method of producing lenses with bilateral hydrogel layers differing in depth or composition.

FIG. 13 is a table illustrating bioconjugation reactions that can be used in some embodiments.

FIG. 14 illustrates linker structures that can be used in some embodiments.

### DETAILED DESCRIPTION

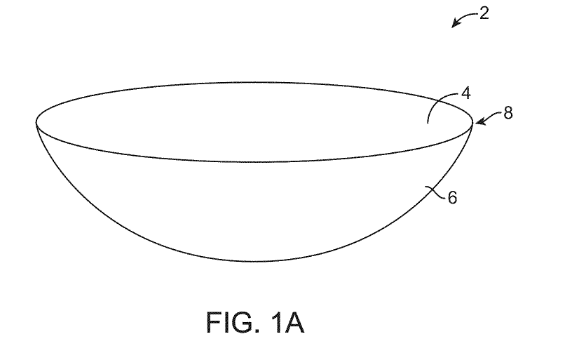

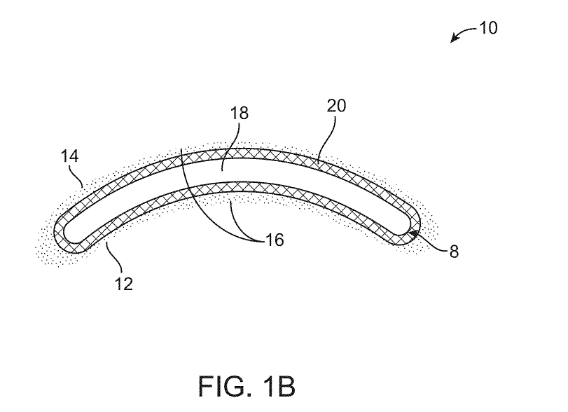

As shown in FIG. 1A, a contact lens 2 may be generally understood as having a body with a concave surface 4 and a convex surface 6. The lens body may include a periphery or a perimeter 8 between the surfaces. The periphery may also include a circumferential edge between the surfaces.

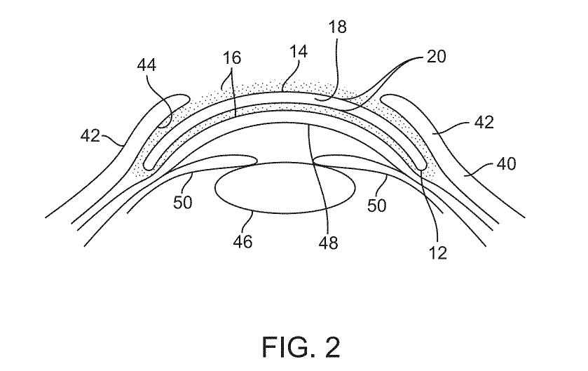

The concave surface 4 may also be referred to as a posterior surface and the convex surface 6 may also be referred to as an anterior surface, terms that refer to respective position when worn by a user. In practice, the concave surface of the lens is adapted to be worn against or adjacent to an ophthalmic surface. When worn the concave surface may lie against a user's corneal surface 48 (see FIG. 2). The convex surface is outward-facing, exposed to the environment when the eye 40 is open. When the eye 40 is closed, the convex surface is positioned adjacent or against the inner conjunctival surface 44 of the eyelids 42 (see FIG. 2).

Because the convex and concave surfaces of a lens may be placed against or adjacent ophthalmic tissue such as the corneal surface, the properties of the surfaces can greatly affect a user's comfort and wearability of the lens as described above. For example, the lens may disrupt the tear film 16 of the eye 40 causing symptoms associated with dry eye. As such, embodiments described herein provide for a coated contact lens having a hydrophilic polymer layer applied on at least one of the lens's surfaces to improve the lens's wettability and wearability with minimal tear film disruption.

In one embodiment, the contemplated coated contact lens includes a core or bulk material with at least one surface having a hydrophilic polymer layer. In some cases, the hydrophilic layer is adapted for placement against an ophthalmic surface. The hydrophilic layer may cover a portion of the lens core surface. Alternatively, the hydrophilic layer may completely or substantially completely cover the core surface.

In other variations, more than one core surface has a hydrophilic layer. For example, both the concave and the convex surfaces of the lens may be coated by a hydrophilic polymer layer. Each hydrophilic layer on either concave or convex surfaces may independently completely or partially cover respective surfaces. In some cases the layer on each side of the core form a contiguous hydrophilic layer across both surfaces.

In additional variations, the hydrophilic polymer layer is formed from a cross-linked hydrogel polymer network having one or more cross-linked species. The hydrophilic polymer network may be partially cross-linked or substantially fully cross-linked. In some variations, the hydrophilic polymer is cross-linked to approximately 95% end group conversion.

Referring to FIG. 1B, a cross-section of an exemplary embodiment of a coated contact lens 10 is shown. Coated contact lens 10 includes a lens core 18 and a hydrophilic polymer layer 20 attached to the core 18. As shown, a hydrophilic polymer layer 20 surrounds the core 18. Both the concave and convex surfaces 12, 14 are coated by the same hydrophilic polymer layer 20 on both sides of the lens 18 with the hydrophilic polymer layer 20 extending to the peripheral edge 8 of the core 10. As shown, the outer hydrophilic layer 20 is substantially contiguous through or across a circumferential edge portion 18.

Referring to FIG. 2, the coated contact lens 10 of FIG. 1B is positioned in a user's eye 40. The eye 40 is shown with eye lens 46 and iris 50. The concave surface 12 of the lens 10 is disposed and centered on the cornea. The convex surface 14 of the lens 10 is directed outwardly, facing the environment when the eye 40 is open. When the eyelid 42 close, the convex surface 14 is adjacent to the inner or conjunctival surface 44 of the eyelid 42. As the eyelids 42 open and close the conjunctival surface 44 slides across the convex surface 14 of the lens 10.

When placed on the cornea, the hydrophilic layer 20 of the contact lens 10 interacts with the natural tear film 16 of the eye 40. The contact lens 10 may be positioned within the tear film 16 and/or substantially reside within the aqueous layer of the tear film 16 that covers the eye 40. In some cases, the lens 10 is immersed in the tear film 16. The hydrophilic layer may be adapted to minimize disruption of the tear film by the contact lens.

A. Hydrophilic Polymer Layer

As used herein, the term “hydrophilic polymer layer” or “hydrophilic coating layer” may refer to a single continuous layer or various coated portions on the lens core.

Although shown in FIG. 1B as a single hydrophilic layer covering both sides of the lens core, it is to be appreciated that in some cases, only a portion of the lens (e.g. a single surface or a part of a surface) may be coated by a hydrophilic polymer layer. In some cases, the hydrophilic layer may only coat one of the core surfaces such as the concave surface. Moreover, the layer may not coat the entire area of the surface.

Additionally, other contemplated embodiments may include two or more noncontiguous hydrophilic polymer layers. For example, a first hydrophilic polymer layer may at least partially cover the concave surface while a second hydrophilic polymer layer may at least partially cover the convex surface. Unlike the embodiment depicted in FIG. 1B, the first and second hydrophilic polymer layer may not touch or share a boundary with one another.

In certain embodiments, the arrangement between the lens core and the surrounding hydrogel or hydrophilic layer may be understood as a layered structure with a hydrophilic polymer layer attached to an outer surface of a lens core layer. The hydrophilic polymer layer may be placed on either of the concave or convex surfaces. In some variations, the hydrophilic layer may only cover a portion of the lens core layer.

In other cases, the arrangement may include a first hydrophilic polymer layer on one side of the lens core layer, a second hydrophilic polymer layer on another side of the lens core layer. The core layer being a middle layer between the two hydrophilic polymer layers. The first and second layers may share a boundary (e.g. contiguous layers) or may form separate independent layers (e.g. noncontiguous layers).

Additionally, the hydrophilic layer may have relatively uniform dimensions, compositions, and mechanical properties throughout. Referring to FIG. 1B, the hydrophilic layer 20 has a substantially uniform thickness, water content, and chemical composition throughout the layer. In some embodiments, the hydrophilic layer has a substantially homogeneous composition and a substantially uniform depth and/or thickness.

As can be appreciated, uniformity is not required and may not be desirable for all situations. In some cases, a single layer may include portions having different characteristics including dimensions, composition, and/or mechanical properties. For example, a portion of the layer may have a different thickness than another portion, which may result in varying water content between the two portions.

Similarly, where two or more hydrophilic layers are used, the hydrophilic polymer layers may share or differ in any characteristics. For example, the core material may be asymmetrically layered with the hydrophilic polymer. The depth/thickness of the resulting hydrophilic polymer layers may vary between the layers on opposing sides of the lens substrate. This can result in, for example, different mechanical characteristics between the concave-cornea facing side of the coated contact lens and the outward facing convex face.

In some variations, the average thickness of the hydrophilic polymer layer may range between about 1 nm and about 500 nm. In some embodiments, the hydrophilic coating layer comprises a thickness between about 1 nm to about 50 nm. In particular embodiments, the hydrophilic layer has a thickness of about 100 nm to about 250 nm. In some embodiments, the hydrophilic coating layer comprises a thickness below about 100 nm. In some embodiments, the hydrophilic coating layer comprises a thickness below about 50 nm. In some embodiments, the hydrophilic coating layer comprises a thickness below about 40 nm.

In some embodiments, the thickness of the hydrophilic layer is between about 1 micron and about 200 microns, or between about 1 micron and about 100 microns, or between about 10 microns and about 200 microns, or between about 25 microns and about 200 microns, or between about 25 microns and about 100 microns, or between about 5 microns and about 50 microns, or between about 10 microns and about 50 microns, or between about 10 microns and about 35 microns, or between about 10 microns and about 25 microns, or between about 1 micron and about 10 microns.

In other embodiments, hydrophilic layer has a thickness between about 0.01 microns and about 1 micron, or between about 0.01 microns and about 0.05 microns, or between about 0.05 microns and about 1 micron, or between about 0.02 microns and about 0.04 microns, or between about 0.025 microns and about 0.075 microns, or between about 0.02 microns and about 0.06 microns, or between about 0.03 microns and about 0.06 microns. In an exemplary embodiment, the hydrophilic layer has an average thickness of between about 0.01 microns and about 25 microns, or between about 0.01 microns and about 20 microns, or between about 0.01 microns and about 15 microns, or between about 0.01 microns and about 10 microns, or between about 0.01 microns and about 5 microns, or between about 0.01 microns and about 2.5 microns, or between about 0.01 microns and about 2 microns. In other variations, the hydrophilic layer has an average thickness from about 0.1 microns to about 20 microns, or from about 0.25 microns to about 15 microns, or from about 0.5 microns to about 12.5 microns, or from about 2 microns to about 10 microns.

In further variations, the thickness or depth of the hydrophilic coating layer may also be expressed in terms of the fold-multiple over a layer that could be represented as a molecular monolayer. In some embodiments, the hydrophilic layer has a thickness of that exceeds the nominal thickness of a molecular monolayer by at least five-fold. For example, in some cases the hydrophilic polymer layer is formed from polymer molecules that have a polymer monolayer radius of about 5 nm. The polymer containing hydrophilic polymer layer may have a thickness of about 50 nm, which results in a layer thickness or depth that is approximately 10-fold greater than the polymer monolayer radius.

Without limitation, the thickness of the anterior or posterior surface of a contact lens of the invention can be determined by Scanning Electron Microscopy, AFM or fluorescence microscopy analysis of a cross section of the contact lens in fully hydrated state as described herein. In an exemplary embodiment, the thickness of the anterior or posterior surface is at most about 30% (i.e., 30% or less), or at most about 20% (20% or less), or at most about 10% (10% or less) of the thickness of the inner layer (e.g. core) of the contact lens described in a fully hydrated state. In an exemplary embodiment, the layers forming the anterior and posterior surface of the contact lens described in this paragraph are substantially uniform in thickness. In an exemplary embodiment, these layers merge at the peripheral edge of the contact lens to completely enclose the inner layer of the silicon-containing layer.

Additionally, the hydrophilic layer may be understood to have a volume. In some cases, a first portion of the layer may have first volume V1 and a second portion of the layer may have a second volume V2. The volume may be calculated based on an estimated surface area of the layer. A total volume may also be understood to be the volume of a single hydrophilic layer (e.g. a layer covering the entire lens) or a sum of various layers with corresponding volumes.

Volume calculations may be based on an estimated surface area of approximately 1.25 square centimeters, on each side of the lens core. In some cases, the hydrophilic polymer layer has a volume in the range of about 15 nl to about 1.5 μl. In other variations, a volume range of about 15 nl to about 150 nl corresponds to an enveloping hydrophilic thickness range of about 50 nm to about 500 nm.

Additionally, in some variations, the hydrophilic layer may host an aqueous pool that includes a portion of the tear film pool volume. The total volume of the tear film is estimated to be about 4 μl to about 10 μl. For the purpose of the following calculation, consider an estimated of total tear film volume of about 7.54 μl. Accordingly, in some embodiments, the hydrophilic layer may host an aqueous pool that comprises about from about 0.2% to about 2% of the total tear film pool volume

For water content of the hydrophilic layer, in some embodiments, the water content is between about 70% and about 98% water by weight. In other embodiments, the hydrophilic layer includes between about 85% and about 95% water by weight. Additionally, the water content of the hydrophilic layer may be expressed either by total water content or by a weight/volume percent. The polymer content of the hydrophilic layer may be described also by a weight/volume percent.

The hydrophilic layer may also include a hydrophilic polymer population having one or more subpopulations or species. In some cases, one or more species or subpopulations are cross-linked to form the hydrophilic polymer layer. The hydrophilic polymer layer precursors may be provided in a solution containing the cross-linkable material. Once cross-linked, the one or more species form the hydrophilic polymer coating.

In one variation, the hydrophilic layer includes a first polymer species and a second polymer species that are at least partially cross-linked together to form the hydrophilic layer. Additionally, the polymer species or subpopulation may include linear and/or branched components. A branched species may include a polymer having a branch count ranging from 2-arm to 12-arm branching. In other embodiments, the branched species may include starred branching with about 100 branches or more.

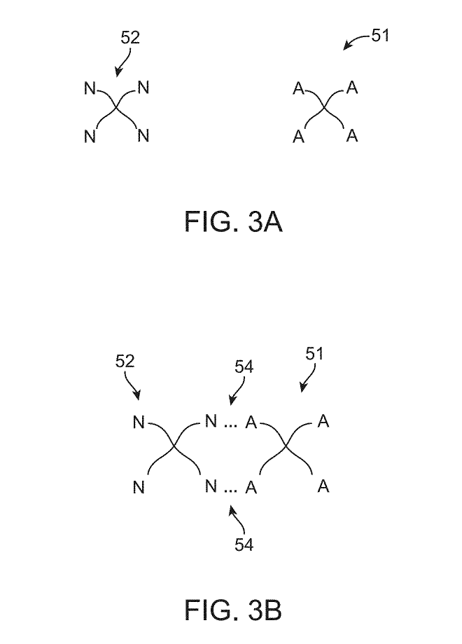

Referring to the FIG. 3A, a first branched polymer species 51 and a second branched polymer species 52 are schematically shown. The first branched polymer species 51 has four branch arms with reactive functional group A. The second branched polymer species 52 is shown having four branch arms with a reactive functional group N. In some embodiments, a reactive moiety A of the first polymer species 51 is adapted to react with a reactive moiety B of the second polymer species 52. The reaction between moieties A and B may form a covalent cross-link between the first and second polymer species. FIG. 3B depicts the first and second species 51, 52 cross-linked by an A-N moiety formed by a reaction between the reactive group A of the first polymer species and a reactive group B of a second polymer species. In some embodiments, the cross-linking action between one or more polymer and/or macromer species forms the hydrophilic polymer layer. For example, cross-linking one or more polymer species in a polymer solution may form a hydrogel with desirable characteristics for coating the lens core.

As can be appreciated, the cross-linking mechanism and/or reaction for a first and second polymer species may include any number of suitable methods known in the art including photochemical or thermal cross-linking. In some cases, cross-linking may occur through nucleophilic conjugate reaction, Michael-type reaction (e.g. 1,4 addition), and/or Click reaction between respective reactive groups on more than one polymer species in the hydrophilic layer.

Any suitable polymers may be used for the hydrophilic polymer population in the hydrophilic layer. In some cases, the polymer population includes species derived from polyethylene glycol (PEG), phosphorylcholine, poly(vinyl alcohol), poly(vinylpyrrolidinone), poly(N-isopropylacrylamide) (PNIPAM), polyacrylamide (PAM), poly(2-oxazoline), polyethylenimine (PEI), poly(acrylic acid), acrylic polymers such as polymethacrylate, polyelectrolytes, hyaluronic acid, chitosan, and dextran.

Additionally, any suitable reactive moieties may be used for the polymer species and subpopulations including reactive functional groups (e.g. reactive nucleophilic groups and electron pair acceptor) that react to form covalent linkages between polymer species or subpopulations to form the hydrophilic polymer layer described.

### 1. Reactive Functional Groups

Reactive functional groups and classes of reactions useful in covalent linking and cross-linking are generally known in the art. In some cases, suitable classes of reactions with reactive functional groups include those that proceed under relatively mild conditions. These include, but are not limited to nucleophilic substitutions (e.g., reactions of amines and alcohols with acyl halides and activated esters), electrophilic substitutions (e.g., enamine reactions) and additions to carbon-carbon and carbon-heteroatom multiple bonds (e.g., Michael reactions and Diels-Alder reactions). These and other useful reactions are discussed, for example, in: March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

### a) Amines and Amino-Reactive Groups

In one embodiment, the reactive functional group is a member selected from amines, such as a primary or secondary amine, hydrazines, hydrazides, and sulfonylhydrazides. Amines can, for example, be acylated, alkylated or oxidized. Useful non-limiting examples of amino-reactive groups include N-hydroxysuccinimide (NHS) esters, sulfo-NHS esters, imidoesters, isocyanates, isothiocyanates, acylhalides, arylazides, p-nitrophenyl esters, aldehydes, sulfonyl chlorides and carboxyl groups.

NHS esters and sulfo-NHS esters react preferentially with the primary (including aromatic) amino groups of the reaction partner. The imidazole groups of histidines are known to compete with primary amines for reaction, but the reaction products are unstable and readily hydrolyzed. The reaction involves the nucleophilic attack of an amine on the acid carboxyl of an NHS ester to form an amide, releasing the N-hydroxysuccinimide.

Imidoesters are the most specific acylating reagents for reaction with the amine groups of e.g., a protein. At a pH between 7 and 10, imidoesters react only with primary amines. Primary amines attack imidates nucleophilically to produce an intermediate that breaks down to amidine at high pH or to a new imidate at low pH. The new imidate can react with another primary amine, thus crosslinking two amino groups, a case of a putatively monofunctional imidate reacting bifunctionally. The principal product of reaction with primary amines is an amidine that is a stronger base than the original amine. The positive charge of the original amino group is therefore retained. As a result, imidoesters do not affect the overall charge of the conjugate.

Isocyanates (and isothiocyanates) react with the primary amines of the conjugate components to form stable bonds. Their reactions with sulfhydryl, imidazole, and tyrosyl groups give relatively unstable products.

Acylazides are also used as amino-specific reagents in which nucleophilic amines of the reaction partner attack acidic carboxyl groups under slightly alkaline conditions, e.g. pH 8.5.

Arylhalides such as 1,5-difluoro-2,4-dinitrobenzene react preferentially with the amino groups and phenolic groups of the conjugate components, but also with its sulfhydryl and imidazole groups.

p-Nitrophenyl esters of carboxylic acids are also useful amino-reactive groups. Although the reagent specificity is not very high, α- and ε-amino groups appear to react most rapidly.

Aldehydes react with primary amines of the conjugate components. Although unstable, Schiff bases are formed upon reaction of the amino groups with the aldehyde. Schiff bases, however, are stable, when conjugated to another double bond. The resonant interaction of both double bonds prevents hydrolysis of the Schiff linkage. Furthermore, amines at high local concentrations can attack the ethylenic double bond to form a stable Michael addition product. Alternatively, a stable bond may be formed by reductive amination.

Aromatic sulfonyl chlorides react with a variety of sites of the conjugate components, but reaction with the amino groups is the most important, resulting in a stable sulfonamide linkage.

Free carboxyl groups react with carbodiimides, soluble in both water and organic solvents, forming pseudoureas that can then couple to available amines yielding an amide linkage. Yamada et al., Biochemistry 1981, 20: 4836-4842, e.g., teach how to modify a protein with carbodiimides.

### b) Sulfhydryl and Sulfhydryl-Reactive Groups

In another embodiment, the reactive functional group is a member selected from a sulfhydryl group (which can be converted to disulfides) and sulfhydryl-reactive groups. Useful non-limiting examples of sulfhydryl-reactive groups include maleimides, alkyl halides, acyl halides (including bromoacetamide or chloroacetamide), pyridyl disulfides, and thiophthalimides.

Maleimides react preferentially with the sulfhydryl group of the conjugate components to form stable thioether bonds. They also react at a much slower rate with primary amino groups and imidazole groups. However, at pH 7 the maleimide group can be considered a sulfhydryl-specific group, since at this pH the reaction rate of simple thiols is 1000-fold greater than that of the corresponding amine.

Alkyl halides react with sulfhydryl groups, sulfides, imidazoles, and amino groups. At neutral to slightly alkaline pH, however, alkyl halides react primarily with sulfhydryl groups to form stable thioether bonds. At higher pH, reaction with amino groups is favored.

Pyridyl disulfides react with free sulfhydryl groups via disulfide exchange to give mixed disulfides. As a result, pyridyl disulfides are relatively specific sulfhydryl-reactive groups.

Thiophthalimides react with free sulfhydryl groups to also form disulfides.

### c) Other Reactive Functional Groups

Other exemplary reactive functional groups include:
(a) carboxyl groups and various derivatives thereof including, but not limited to, N-hydroxybenztriazole esters, acid halides, acyl imidazoles, thioesters, p-nitrophenyl esters, alkyl, alkenyl, alkynyl and aromatic esters;
(b) hydroxyl groups, which can be converted to esters, ethers, aldehydes, etc.;
(c) haloalkyl groups, wherein the halide can be displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) alkenes, which can undergo, for example, cycloadditions, acylation, Michael addition, etc;
(g) epoxides, which can react with, for example, amines and hydroxyl groups;
(h) phosphoramidites and other standard functional groups useful in nucleic acid synthesis and
(i) any other functional group useful to form a covalent bond between the functionalized ligand and a molecular entity or a surface.
   d) Reactive Functional Groups with Non-Specific Reactivities

In addition to the use of site-specific reactive moieties, the present invention contemplates the use of non-specific reactive functional groups. Non-specific groups include photoactivatable groups, for example. Photoactivatable groups are ideally inert in the dark and are converted to reactive species in the presence of light. In one embodiment, photoactivatable groups are selected from macromers of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently preferred. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, hydroxy and nitroarylazides may be preferable since they allow to employ less harmful photolysis conditions for the affinity component than unsubstituted arylazides.

In an exemplary embodiment, photoactivatable groups are selected from fluorinated arylazides. The photolysis products of fluorinated arylazides are arylnitrenes, all of which undergo the characteristic reactions of this group, including C-H bond insertion, with high efficiency (Keana et al., J. Org. Chem. 55: 3640-3647, 1990).

In another embodiment, photoactivatable groups are selected from benzophenone residues. Benzophenone reagents generally give higher crosslinking yields than arylazide reagents.

In another embodiment, photoactivatable groups are selected from diazo compounds, which form an electron-deficient carbene upon photolysis. These carbenes undergo a variety of reactions including insertion into C-H bonds, addition to double bonds (including aromatic systems), hydrogen attraction and coordination to nucleophilic centers to give carbon ions.

In still another embodiment, photoactivatable groups are selected from diazopyruvates. For example, the p-nitrophenyl ester of p-nitrophenyl diazopyruvate reacts with aliphatic amines to give diazopyruvic acid amides that undergo ultraviolet photolysis to form aldehydes. The photolyzed diazopyruvate-modified affinity component will react like formaldehyde or glutaraldehyde.

It is well within the abilities of a person skilled in the art to select a reactive functional group, according to the reaction partner. As an example, an activated ester, such as an NHS ester can be a useful partner with a primary amine. Sulfhydryl reactive groups, such as maleimides can be a useful partner with SH, thiol, groups.

Additional exemplary combinations of reactive functional groups found on a compound of the invention and on a targeting moiety (or polymer or linker) are set forth in Table 1.

TABLE 1

| Chemical | Chemical | |
|---|---|---|
| Functionality 1 | Functionality 2 | Linkage |
| Hydroxy | Carboxy | Ester |
| | Hydroxy | Carbonate |
| | Amine | Carbamate |
| | SO₃ | Sulfate |
| | PO₃ | Phosphate |
| | Carboxy | Acyloxyalkyl |
| | Ketone | Ketal |
| | Aldehyde | Acetal |
| | Hydroxy | Anhydride |
| Mercapto | | Disulfide |
| | Carboxy | Acyloxyalkyl |
| | | Thioether |
| | Carboxy | Thioester |
| | Carboxy | Amino amide |
| | Mercapto | Thioester |
| | Carboxy | Acyloxyalkyl |
| | | ester |
| | Carboxy | Acyloxyalkyl |
| | | amide |
| | Amino | Acyloxyalkoxy carbonyl |
| | Carboxy | Anhydride |
| | Carboxy | N-acylamide |
| | Hydroxy | Ester |
| | Hydroxy | Hydroxymethyl |
| | | ketone ester |
| | Hydroxy | Alkoxycarbonyl oxyalkyl |
| Amino | Carboxy | Acyloxyalkylamine |
| | Carboxy | Acyloxyalkylamide |
| | Amino | Urea |
| | Carboxy | Amide |
| | Carboxy | Acyloxyalkoxycarbonyl |
| | Amide | N-Mannich base |
| | Carboxy | Acyloxyalkyl carbamate |
| Phosphate | Hydroxy | Phosphate oxygen ester |
| | Amine | Phosphoramidate |
| | Mercapto | Thiophosphate ester |
| Ketone | Carboxy | Enol ester |
| Sulfonamide | Carboxy | Acyloxyalkyl sulfonamide |
| | Ester | N-sulfonyl-imidate |

One skilled in the art will readily appreciate that many of these linkages may be produced in a variety of ways and using a variety of conditions. For the preparation of esters, see, e.g., March supra at 1157; for thioesters, see, March, supra at 362-363, 491, 720-722, 829, 941, and 1172; for carbonates, see, March, supra at 346-347; for carbamates, see, March, supra at 1156-57; for amides, see, March supra at 1152; for ureas and thioureas, see, March supra at 1174; for acetals and ketals, see, Greene et al. supra 178-210 and March supra at 1146; for acyloxyalkyl derivatives, see, PRODRUGS: TOPICAL AND OCULAR DRUG DELIVERY, K. B. Sloan, ed., Marcel Dekker, Inc., New York, 1992; for enol esters, see, March supra at 1160; for N-sulfonylimidates, see, Bundgaard et al., J. Med. Chem., 31:2066 (1988); for anhydrides, see, March supra at 355-56, 636-37, 990-91, and 1154; for N-acylamides, see, March supra at 379; for N-Mannich bases, see, March supra at 800-02, and 828; for hydroxymethyl ketone esters, see, Petracek et al. Annals NY Acad. Sci., 507:353-54 (1987); for disulfides, see, March supra at 1160; and for phosphonate esters and phosphonamidates.

The reactive functional groups can be chosen such that they do not participate in, or interfere with, the reactions necessary to assemble the reactive ligand analogue. Alternatively, a reactive functional group can be protected from participating in the reaction by the presence of a protecting group. Those of skill in the art will understand how to protect a particular functional group from interfering with a chosen set of reaction conditions. For examples of useful protecting groups, see Greene et al., Protective Groups in Organic Synthesis, John Wiley & Sons, New York, 1991.

Generally, prior to forming the linkage between the compound of the invention and the targeting (or other) agent, and optionally, the linker group, at least one of the chemical functionalities will be activated. One skilled in the art will appreciate that a variety of chemical functionalities, including hydroxy, amino, and carboxy groups, can be activated using a variety of standard methods and conditions. For example, a hydroxyl group of the ligand (or targeting agent) can be activated through treatment with phosgene to form the corresponding chloroformate, or p-nitrophenylchloroformate to form the corresponding carbonate.

In an exemplary embodiment, the invention makes use of a targeting agent that includes a carboxyl functionality. Carboxyl groups may be activated by, for example, conversion to the corresponding acyl halide or active ester. This reaction may be performed under a variety of conditions as illustrated in March, supra pp. 388-89. In an exemplary embodiment, the acyl halide is prepared through the reaction of the carboxyl-containing group with oxalyl chloride. The activated agent is combined with a ligand or ligand-linker arm combination to form a conjugate of the invention. Those of skill in the art will appreciate that the use of carboxyl-containing targeting agents is merely illustrative, and that agents having many other functional groups can be conjugated to the ligands of the invention.

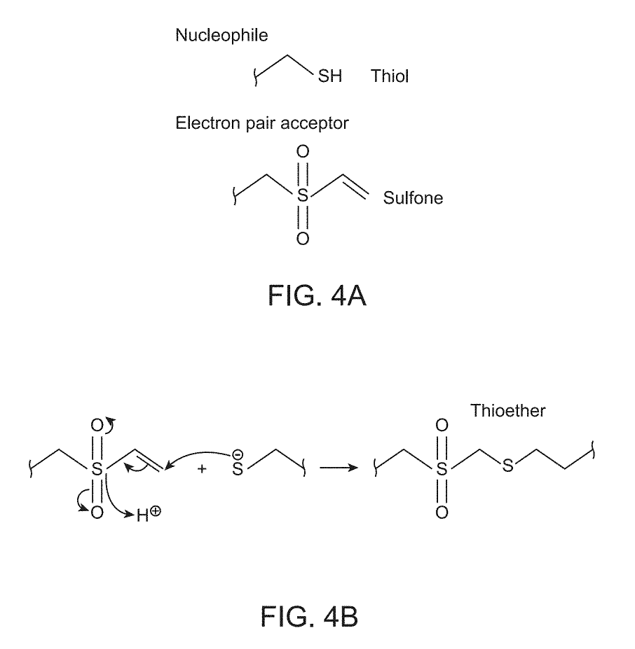

Referring to FIG. 4A, in some embodiments, the reactive functional groups include thiol and sulfonyl moieties. The reactive nucleophilic group may be a thiol group adapted to react to a sulfonyl group that functions as an electron pair accepting moiety. Where a first polymer species contains a reactive thiol group and a second polymer species contains a reactive sulfonyl group, the cross-linkage between the first and second species may be formed through a thioether moiety (FIG. 4B).

In other variations, one or more polymer species in the hydrophilic layer are covalently linked through a sulfonyl moiety such as, but not limited to, an alkylene sulfonyl moiety, a dialkylene sulfonyl moiety, an ethylene sulfonyl moiety, or a diethylene sulfonyl moiety. In further variations, one or more polymer species in the hydrophilic layer are covalently linked through a sulfonyl moiety and a thioether moiety, or an alkylene sulfonyl moiety and a thioether moiety, or a dialkylene sulfonyl moiety and a thioether moiety, or an ethylene sulfonyl moiety and a thioether moiety, or a diethylene sulfonyl moiety and a thioether moiety.

In further variations, the one or more polymer species in the hydrophilic layer are covalently linked through an ester moiety, or alkylene ester moiety, or an ethylene ester moiety, or a thioether moiety, or an ester moiety and a thioether moiety, or an alkylene ester moiety and a thioether moiety, or an ethylene ester moiety and a thioether moiety.

In some embodiments, the ratio of the reactive subpopulations in the hydrophilic polymer population is approximately 1 to 1. In other embodiments, the concentration of one of the subpopulations or species exceeds another species by about 10% to about 30%. For example, the concentration of a polymer species with an electron pair accepting moiety may exceed another polymer species with a reactive nucleophilic group.

Additionally, where the concentration of a first and second polymer species are approximately 1 to 1, the relative number of reactive moieties for each species may be approximately the same or different. For example, a polymer species may have more sites having an electron pair accepting moiety compared to the number of reactive sites on the other polymer species carrying the nucleophilic group. This may be accomplished, for example, by having a first branched polymer species having more arms with reactive electron pair accepting sites compared to a second polymer species carrying the nucleophilic moiety.

### 2. Polymer-Containing Hydrophilic Layer

In some embodiments, the polymers in the hydrophilic layer comprise polyethylene glycol (PEG). The PEG may include species that have a molecular weight of between about 1 kDa and about 40 kDa. In particular embodiments, the PEG species have a molecular weight of between about 5 kDa and about 30 kDa. In some embodiments, the hydrophilic polymer population consists of a species of polyethylene glycol (PEG). In other variations, the weight average molecular weight M_{w} of the PEG polymer having at least one amino or carboxyl or thiol or vinyl sulfone or acrylate moiety (as a hydrophilicity-enhancing agent) can be from about 500 to about 1,000,000, or from about 1,000 to about 500,000. In other embodiments, the hydrophilic polymer population comprises different species of PEG.

In some cases, the polymer includes subunits of PEG. In some variations, the subunits of the polymers of the PEG-containing layer of the contact lens are at least about 95%, or at least about 96%, or at least about 97%, or at least about 98%, or at least about 99% or at least about 99.5% polyethylene glycol.

In some cases, the water content of the PEG-containing hydrophilic layer is between about 80% and about 98% water by weight. In other embodiments, the hydrophilic layer includes between about 85% and about 95% water by weight.

The PEG-containing hydrophilic layer may include a PEG hydrogel having a swelling ratio. To determine swelling ratio, the PEG-hydrogel can be weighed immediately following polymerization and then immersed in distilled water for a period of time. The swollen PEG hydrogel is weighed again to determine the amount of water absorbed into the polymer network to determine the swelling ratio. The mass fold increase an also be determined based on this comparison before and after water swelling. In some embodiments, the PEG-containing layer has a mass fold increase of less than about 10%, or of less than about 8%, or of less than about 6%, or of less than about 5%, or of less than about 4%, or of less than about 3%, or of less than about 2%, or of less than about 1%. In some cases, the mass fold increase is measured by weighing the hydrogel when wet and then dehydrating it and weighing it again. The mass fold increase is then the swollen weight minus the dry weight divided by the swollen weight. For the hydrophilic layer as opposed to a bulk hydrogel, this could be accomplished by coating a non-hydrated substrate and then performing mass change calculations.

In another aspect, the invention provides for a hydrophilic layer with two cross-linkable PEG species. The first PEG species may include a reactive functional group adapted to react to another reactive functional on the second PEG species. Any of the described functional groups (e.g. previous section (A)(1)) may be suitable for forming a cross-linkage between the first and second PEG species.

In some cases, the first PEG species includes an electron pair accepting moiety and the second PEG species may include a reactive nucleophilic moiety. Once cross-linked through a reaction between the electron pair accepting and nucleophilic moieties, the PEG polymer network forms a hydrogel with a water content or concentration. The PEG hydrogel may serve as the hydrophilic layer coating a lens core to provide improved wettability, wearability, and/or reduced tear film disruption.

### 3. Active Agents

The hydrophilic polymer layer may include active agents such as any one or more of a medicinal agent, UV-absorbing agent, a visibility tinting agent, an antimicrobial agent, a bioactive agent, silver, a leachable lubricant, a leachable tear-stabilizing agent, or any mixture thereof. The substances and materials may be deposited on the contact lenses to augment the interaction of a contact lens with the ocular region. These substances may consist of polymers, drugs, or any other suitable substance and may be used to treat a variety of ocular pathologies including but not limited to dry eye disease, glaucoma, allergies, corneal ulcers, scleritis, keratitis, iritis, and corneal neovascularization.

### 4. Interpenetration Polymer Network

The outer hydrogel network may also consist of interpenetrating polymer networks (or semi-interpenetrating polymer networks) formed in either simultaneous or sequential polymerization steps. For example, upon forming the initial outer hydrophilic coating layer, the layer can be swollen in a monomer solution such as acrylic acid along with a crosslinker and initiator. Upon exposure to UV light, a second interpenetrating network will form. The double network confers additional mechanical strength and durability while maintaining high water content and high wettability.

Hydrophilic layers, such as PEG were not considered to have good long term stability. In co-owned application Ser. No. 13/975,868 filed on Aug. 26, 2013, PEG layers formed on soft core lenses were analyzed with accelerated aging studies. The aging studies indicated that the PEG layers had better than expected shelf life and stability. The longevity of the coating with longer wear and more rigorous cleaning was unexpected. Additional testing has shown that the coating processes work well with RGP and hybrid RGP lenses. In addition the coatings have demonstrated a suitable shelf life for RGP and hybrid RGP lenses even with exposure to the more rigorous cleaning processes associated with those lenses. Additional details for the testing of the coatings through autoclave sterilization and accelerated aging tests are detailed in the examples.

### B. Lens Core

A suitable contact lens core includes a lens with high silicone content. The lens core may consist substantially entire of pure silicone, i.e. the core comprises about 100% silicone by weight. In other cases, the lens core, base, or substrate comprises about 50% to about 100% of silicone by weight. In some cases, the substrate or core comprises about 80 to 98% silicone by weight.

In an exemplary embodiment, the silicone-containing layer is a silicone elastomer. In some cases, the silicone-containing layer or core of the coated contact lens is a copolymer of multiple types of silicone.

In an exemplary embodiment, the silicone-containing layer is comprised of a silicone with a low viscosity to allow injection molding of the core lens.

In another embodiment the silicone core can also be made using multifunctional siloxane macromers containing thiol and alkene functionalities and taking advantage of the rapid click type “thiol-ene” reaction. For example, vinyl terminated siloxane combined with (mercaptopropyl)methylsiloxane-dimethylsiloxane copolymers containing from 2-99 mol % (mercapto-propyl) methylsiloxane and exposed to UV light will crosslink to form silicone elastomers. To improve molding of the materials, an additional difunctional mercaptosiloxane is added to the mix which will serve to increase the molecular weight between crosslinks, and therefore elasticity of the material, without increasing the viscosity of the prepolymer mix. The thiol-ene silicone elastomer can also be tailored by adjusting the stoichiometry of the underlying mixture to yield free thiols on the surface that can then be used to react with the crosslinked hydrophilic polymer coating.

In another embodiment, the lens core may contain a silicone-hydrogel (SiHy) where the core is more hydrophilic than a pure silicone core but less hydrophilic than a pure hydrogel. In such cases, the SiHy lens core can be coated by the described hydrophilic polymer layers to improve wettability and wearability of the lens core. In other variations, the core comprises about 10% to about 20% of silicone by weight. In some cases, the silicone-containing layer or core of the coated contact lens is lotrafilcon, balafilcon, galyfilcon, senofilcon, narafilcon, omafilcon, comfilcon, enfilcon, or asmofilcon.

Alternatively, a non-silicone based core may be used as the substrate for the coating. For example, an oxygen permeable lens made from a non-silicone material may also be coated with the described hydrophilic layer.

In an exemplary embodiment, the thickness of the core or core layer is from about 0.1 microns to about 200 microns, or from about 1 microns to about 150 microns, or from about 10 microns to about 100 microns, or from about 20 microns to about 80 microns, or from about 25 microns to about 75 microns, or from about 40 microns to about 60 microns.

### C. Attachment of Hydrophilic Layer to Core

Another aspect of the invention provides for a coated contact lens with hydrophilic polymer layer that is covalently linked and attached to the core. The covalent linkage between the hydrophilic layer and the core may be understood to be a linking moiety that is covalently disposed between the lens core and the hydrophilic layer. In some cases, the linking moiety covalently attaches the hydrophilic layer to an outer surface of the lens core.

In some embodiments, the linking moiety may include any of the reactive functional groups described in at least section (A)(1). In further variations, the linking moiety may be a resultant moiety formed from a reaction between one or more of the reactive functional groups described in at least section (A)(1). For example, the linking moiety may include an electron pair accepting group such as a Michael-type Michael-Type electron pair accepter (e.g. sulfone group) on a polymer species in the hydrophilic layer that reacts to covalently attach the hydrophilic polymer layer to the core.

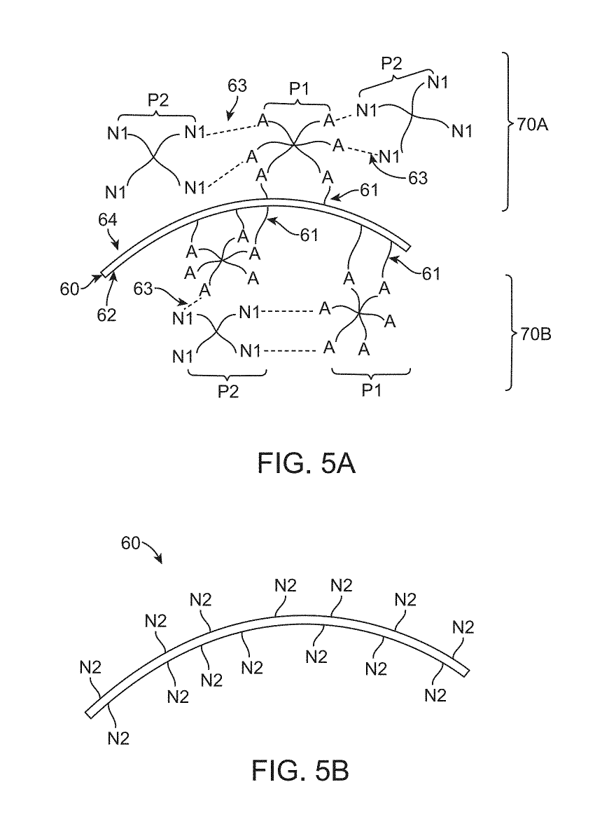

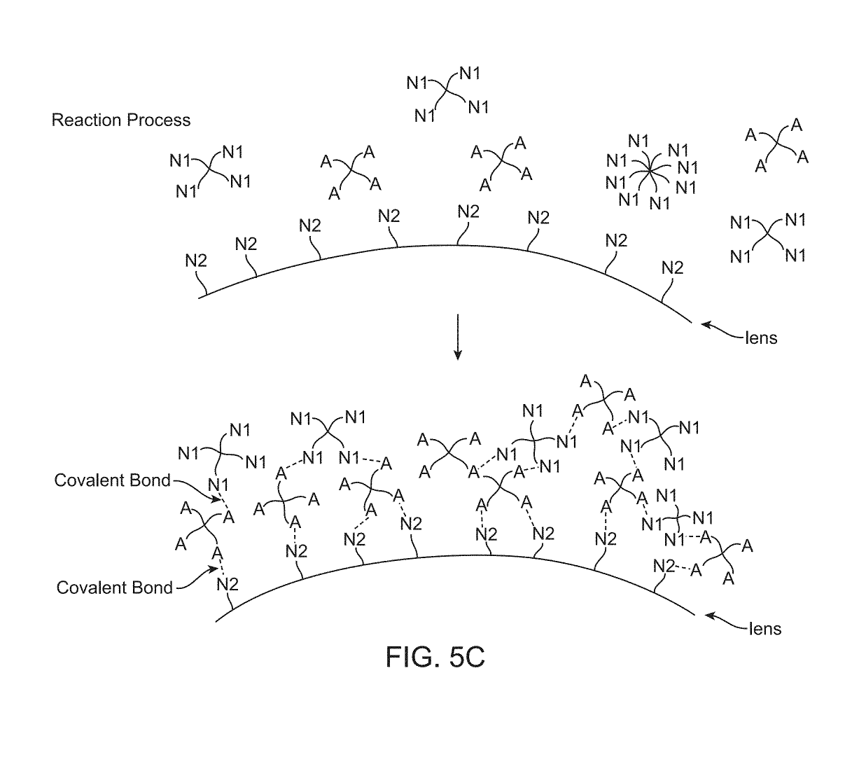

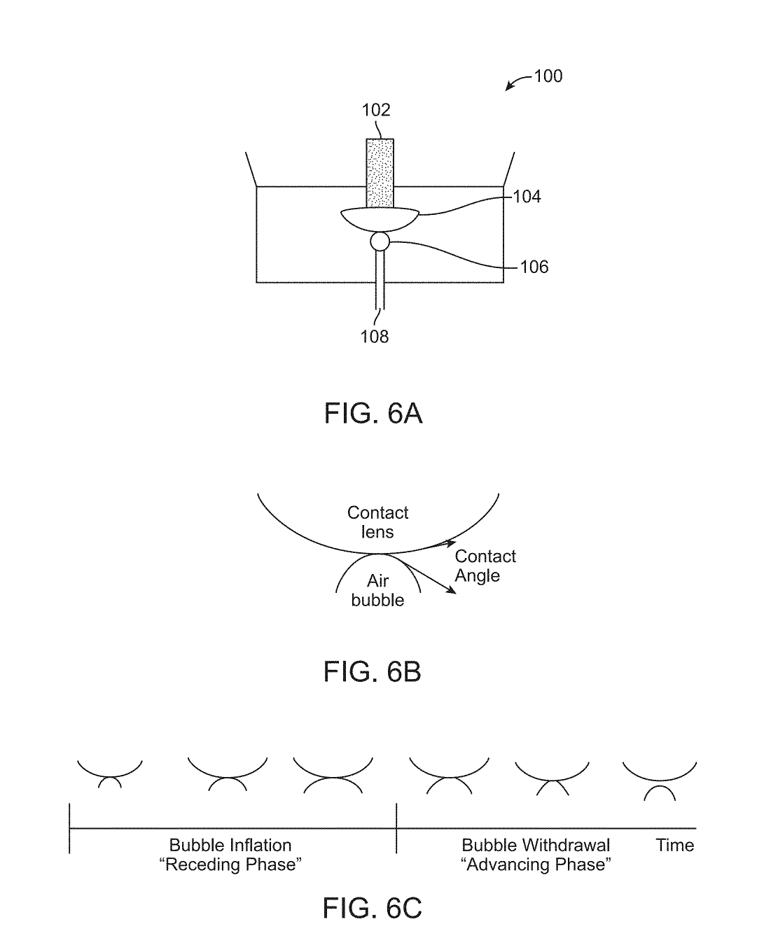

Advantageously, the hydrophilic polymer layer may be attached to the core through similar reactions utilized to cross-link the hydrophilic polymer layer. Referring to FIGS. 5A-5C, the hydrophilic polymer layer includes a first polymer species P1 having a reactive group A and second polymer species P2 with a reactive group Ni. As described earlier, the hydrophilic polymer layer may be formed by cross-linking the first polymer species and the second polymer species through a reaction between reactive group A and Ni. As shown in FIG. 5A cross-linkages 63 covalently link the first and second species to form the first hydrophilic polymer layer 70A on the convex surface 64 and the second hydrophilic polymer layer 70B on the concave surface 62 of the lens core 60.

Referring still to FIG. 5A, the first polymer species also forms a covalent linkage 61 with the outer surface of the core. As shown, the covalent linkage is formed through the reactive group A of the first polymer species P1 and the core surface. In some embodiments, the reactive group A on the first polymer species P1 reacts to (1) crosslink the polymer species in the hydrophilic polymer layer and (2) attach the formed hydrophilic polymer layer to the core. In such cases, this permits a first portion of the A moieties to react with the N1 moieties and a second portion of A moieties to react with the core surface. In some cases, the concentration of the first polymer species P1 and/or the number of available reactive A moieties of the first polymer species exceeds the corresponding concentration of the second polymer species and/or available reactive N1 moieties.

Referring to FIG. 5B, the lens core may include a reactive moiety N2. Reactive moiety N2 may be adapted to react with reactive groups of polymer species in the hydrophilic polymer layer. In some cases, the reactive moiety N2 only reacts to one of the polymer species. Referring to FIG. 5C, reactive moiety N2 reacts with reactive group A on the first species P1 to form a covalent attachment between the hydrophilic polymer layer and the core.

As can be appreciated, the reaction for attaching the hydrophilic polymer layer to the core may include any number of suitable methods known in the art including those described in at least section (A)(1). In some cases, covalent linking occurs through nucleophilic conjugate reaction, Michael-type reaction (e.g. 1,4 addition), and/or Click reaction between respective reactive groups on more than one polymer species in the hydrophilic layer.

In some cases, the reactive A group is an electron pair acceptor and the reactive groups N1 and N2 are reactive nucleophilic groups. N1 and N2 may be the same or different reactive groups. Continuing with the example shown in FIGS. 5A-5C, the hydrophilic polymer layer is formed by a first reaction between the reactive A group and reactive nucleophile N1. Additionally, the hydrophilic polymer layer is covalently attached to the core through a second reaction between the reactive A group and nucleophile N2. The two reactions may occur simultaneously or near simultaneously in the same reaction vessel.

Where the reactive functional groups include thiol and sulfonyl moieties, the reactive A group may be a sulfonyl group on a first PEG macromer. The sulfone moiety functions as an electron pair accepting moiety on the first PEG macromer. The reactive nucleophiles N1 and/or N2 may be a thiol group (see FIG. 4A). For the first reaction, the first and second PEG macromers form a cross-link through the reactive thiol and sulfonyl groups, which can results in a thioether moiety (see FIG. 4B). Where the N2 nucleophile on the core is also thiol, a thioether may also be formed by a reaction between the sulfonyl moiety on the first PEG macromer and the N2 on the surface of the lens core.

As can be appreciated, the nucleophilic group (or other type of reactive group) on the core does not need to be the same as the reactive groups in the hydrophilic polymer layers. However, utilizing the same reactive groups may provide some advantages such as controllability and predictability of the respective reactions.

In other variations, the hydrophilic polymer layer are covalently linked to the lens core through a sulfonyl moiety such as, but not limited to, an alkylene sulfonyl moiety, a dialkylene sulfonyl moiety, an ethylene sulfonyl moiety, or a diethylene sulfonyl moiety. In further variations, the hydrophilic polymer layer is covalently attached to the core through a sulfonyl moiety and a thioether moiety, or an alkylene sulfonyl moiety and a thioether moiety, or a dialkylene sulfonyl moiety and a thioether moiety, or an ethylene sulfonyl moiety and a thioether moiety, or a diethylene sulfonyl moiety and a thioether moiety.

In further variations, the hydrophilic polymer layer is covalently attached to the core through an ester moiety, or alkylene ester moiety, or an ethylene ester moiety, or a thioether moiety, or an ester moiety and a thioether moiety, or an alkylene ester moiety and a thioether moiety, or an ethylene ester moiety and a thioether moiety.

In further embodiments, the linkage between the core lens and the hydrophilic layer is covalent, to the particular exclusion of any other form of chemical bond or association. For example, a hydrophilic coating layer as described may be bound to the surface of a hydrophobic lens core by a chemical bond that consists of a covalent bond.

In further embodiments, the core contact lens monomer mix contains activating components that enable covalent attachment to the hydrophilic layer in the absence of plasma.

Covalent attachment of a dense, crosslinked polymer layer typically requires a high density of chemical reactive groups at the interface. However, this approach is not feasible for contact lenses because the core lens properties must be maintained and therefore only small concentrations of chemically reactive activator can be added directly to the lens monomer mix. To overcome this limitation, prior art (Qiu) used layer by layer dip coating to electrostatically bind a polymer layer with a high density of chemical reactive groups to the core lens. A crosslinked hydrophilic layer was then covalently attached to the electrostatically bound polymer layer that contained the high density of reactive sites.

The need for a high number of reactive sites at the interface is due in part to excluded volume effects at the lens surface. Excluded volume refers to the fact that polymer molecules are inhibited from moving in the volume occupied by other molecules. In dilute solutions and good solvents, polymer molecules will resist approaching each other such that the center of the approaching molecule is excluded from a volume equal to eight times the volume of the molecule.

When polymer solutions interact with surfaces, there is also an excluded volume at the interface. This excluded volume is a function of the properties of the interface, solvent, and polymer system. For silicone hydrogel contact lenses, the surface is hydrophobic and therefore hydrophilic polymers in aqueous solutions result in large excluded volumes at the interface. Essentially this means that approaching polymer molecules are excluded from a thin layer near the surface due to the excluded volume effects. Therefore, because of this physical force, including only a low density of chemical reactive sites in the lens monomer mix will not enable covalent binding of a crosslinked hydrophilic layer to the lens surface.

To overcome the excluded volume effect and facilitate direct covalent attachment of the hydrophilic layer to the lens core, we have developed a method that utilizes a combination of a chemical activator and a physical activator. The activating molecules are dual functional molecules that covalently react with the lens monomer mix and also provide an additional functional group. The chemical activator provides a complementary chemical reactive group that covalently reacts with the hydrophilic polymer solution. The physical activator introduces a physical force that overcomes the excluded volume effect at the interface. In isolation neither activator is sufficient to produce covalently attached, crosslinked hydrophilic layers. However, in combination, the activators work synergistically and enable surface activation at low activator concentrations.

The system in this case consists of the hydrophilic polymer to be attached, the contact lens, and the solvent. To alter the physics of the system and overcome the excluded volume effect at the interface, any of these three parameters can be manipulated. Hydrophilic polymer properties are constrained by the desired on eye performance and therefore only minimal adjustments can be made to this component. Solvent properties are also constrained due to the need for the hydrophilic polymer solubility to facilitate coating. Therefore polymer/solvent properties such as solvent quality may be utilized to optimize covalent attachment. In a preferred embodiment, physical activation of the core lens the primary force in overcoming excluded volume effects in the system.

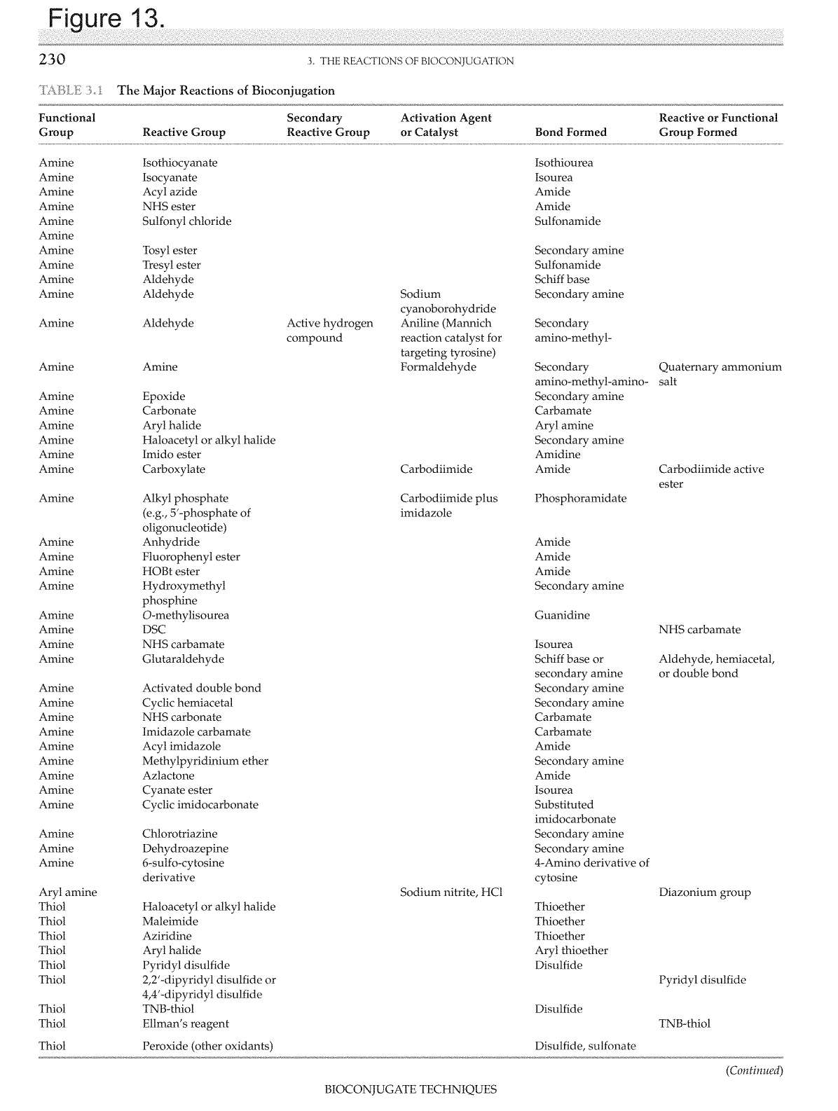

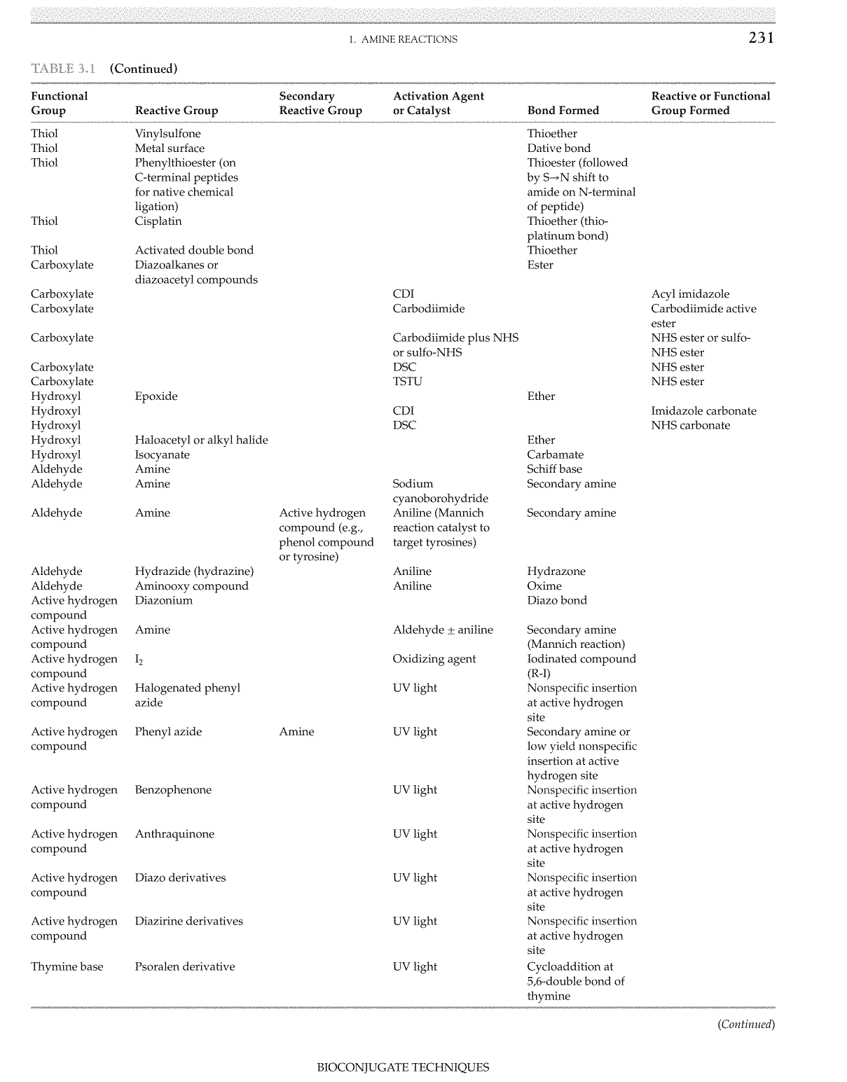

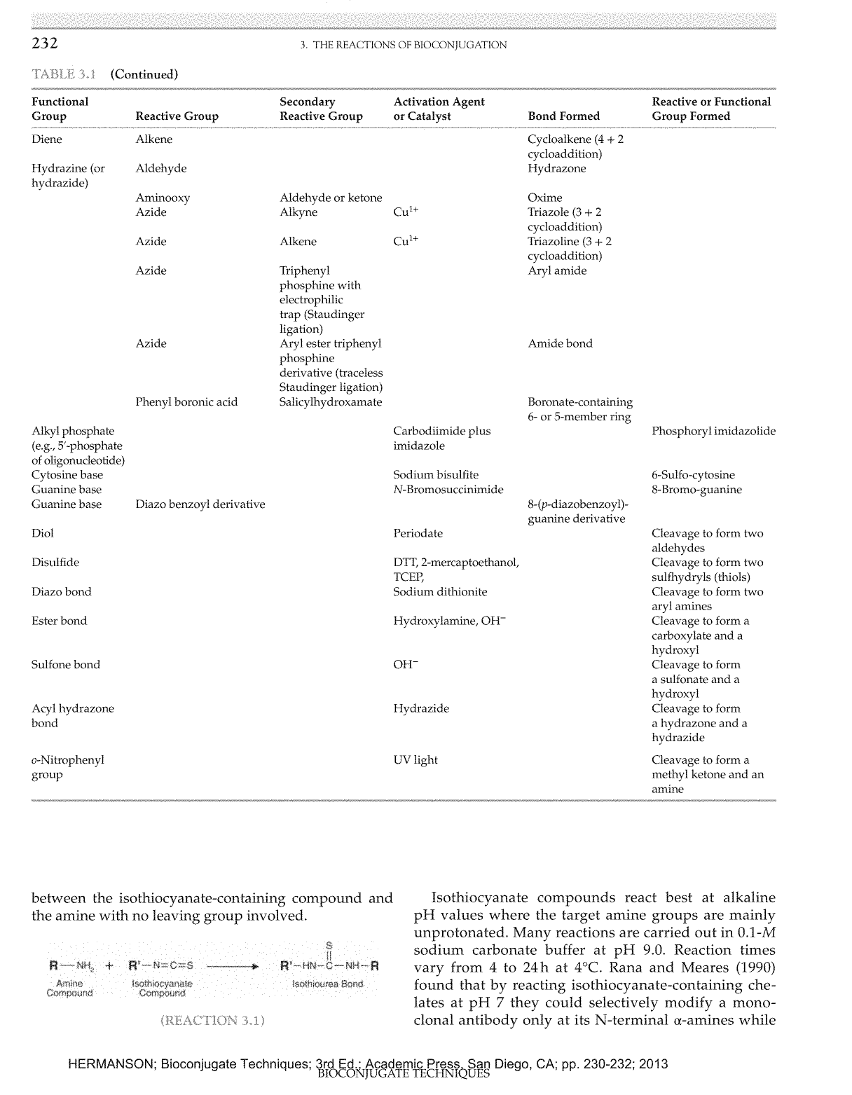

The chemical activator molecule may be used to provide surface reactive moieties for covalent attachment of the hydrophilic polymer layer. The reactive moieties should be reactive under relatively mild “click-type” reactions. A list of suitable reactive pairs is given in FIG. 13. In addition, reactions between alkynes and azides may be used, especially reactions that take advantage of strained alkynes to eliminate the need for copper catalysts, for example dibenzocyclooctyne-amine. Reactions between double bonds and thiols that are accelerated by exposure to UV energy may also be utilized. These reactive pairs are selected in conjunction with the reactive pairs selected for the hydrophilic layer as well as the polysaccharide analogue layer such that the reactive groups for all components involved are complementary.

The lens may be chemically activated by following several different approaches. First, the lens may be activated through incomplete radical polymerization of the lens monomers thus yielding double bonds, for example acrylate or allyl bonds, that may be subsequently reacted with complementary moieties on the hydrophilic polymers.

The physical activator molecule may be used to introduce a physical force in the system that overcomes the excluded volume effect at the interface between the contact lens and the reactive polymer solution. The physical activator may introcuce electrostatic forces that pull polymers to the surface, for example introduction of carboxylic acid moieties are negatively charged and can result in electrostatic forces between the polymers in solution and the contact lens surface. The physical activator may also be a molecule with phase change behavior that can trigger changes in surface energy of the interface. For example n-isopropyl acrylamide undergoes a phase change at 35C and this trigger temperature can be used to alter the polymer physics of the system in a controlled manner.

The lens may also be chemically and physically activated through addition of monomeric units that contain moieties for reaction. For example addition of allyl methacrylate or 2-aminoethyl methacrylate hydrochloride yields allyl and amino groups. Addition of methacrylic acid yields carboxylic acid groups. Other methacrylate monomers containing reactive moieties may also be used to produce lenses with available chemical functional groups.

In a preferred embodiment, the activator molecule consists of a heterobifunctional linker molecule with a UV reactive moiety (or component that reacts with the base lens mixture) as well as a reactive moiety that can later be utilized for reaction with the hydrophilic polymer layer (groups as described in FIG. 13).

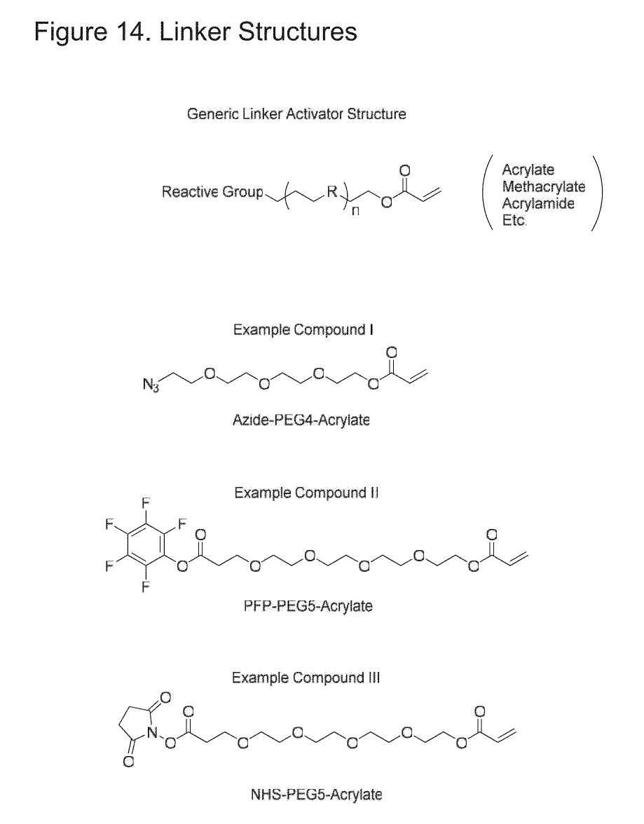

Activator molecules may consists of hydrophilic backbone linkers or surfactant backbone linkers. The hydrophilic nature of the backbone results in migration of the linking moiety to the surface upon placing the lens in an aqueous environment, however silicone hydrogel monomer mixes are not hydrophilic and in order to enable solubility the linker may require surfactant like properties. For short PEG linker lengths the molecules may not need surfactant character to be solubilized. Therefore the required concentration of activator in the monomer mix is minimized and other undesirable impacts on the lens properties are minimized. Examples of linker molecule structure are shown in FIG. 14. In a preferred embodiment the linker consists of poly(ethylene oxide) repeat units, with the number of repeats between 3-10. In a preferred embodiment, the linker consists of a block copolymer. In a preferred embodiment the linker consists of poly(vinyl pyrollidone).

Cleavable bonds may also utilized as a method of producing chemical moieties on the lens surface. For example a bis-acrylamide with a dithiol linkage may be added to the monomer mix and then reduced after lens formation in order to yield free thiol bonds on the surface of the lens. Other examples include protecting groups that are used to prevent reaction during the radical polymerization and can then be cleaved to yield a free functional group, for example fmoc and tboc protected amine groups, or salted amines. Protecting groups may also be used to protect the functional reactive groups on the ends of linkers.

It is unexpected that functional groups will remain on the surfaces of standard lens formulations because the radical polymerization process typically quenches all of the reactive groups present in the lens mixture. The approaches described here provide a method for including reactive groups that remain following radical polymerization.

For producing molded lenses, the reactive groups introduced into the lens formulation may remain reactive for between 1 day and 6 months. For producing lathe cut lenses, activator will be included in button material and activator must remain stable for longer time periods, potentially up to 1 year.

Functional groups for reaction to the hydrophilic layer may also be produced through layer by layer modification of the lens molds or through layer by layer dip coating of the lens in polymer solutions that contain functional reactive moieties.

### D. Multi-Layer Contact Lens

In some embodiments, the coated contact lens contemplated herein is a layered lens with a hydrophilic polymer layer on a silicone-containing layer. Some variations provide for a silicone-containing layer and a first hydrophilic polymer-containing layer, wherein the first hydrophilic polymer containing layer and the silicon-containing layer are covalently attached to one another, and the contact lens has a layered structural configuration. In an exemplary embodiment, the contact lens does not comprise a second silicone-containing layer. In other embodiments, the contact lens does not comprise a second hydrophilic polymer-containing layer. In another embodiment, the contact lens does not comprise either a second silicone-containing layer or a second hydrophilic polymer-containing layer. In an exemplary embodiment, the contact lens comprises an anterior surface and a posterior surface wherein the anterior surface is the first hydrophilic polymer-containing layer and the posterior surface is the silicone-containing layer. In an exemplary embodiment, the contact lens comprises an anterior surface and a posterior surface wherein the anterior surface is the silicone-containing layer and the posterior surface is the first hydrophilic polymer-containing layer.

In an exemplary embodiment, the layer which forms the anterior surface and the layer which forms the posterior surface of the contact lens are of substantially the same thickness. In other cases, the layers may independently have any suitable thickness, including the thickness described above for either the hydrophilic coating layer or the core.

In another aspect, the invention provides a contact lens comprising a silicone-containing layer, a first hydrophilic polymer containing layer and a second hydrophilic polymer containing layer, wherein the first hydrophilic polymer containing layer and the silicone-containing layer are covalently attached to one another, and the second hydrophilic polymer containing layer and the silicone-containing layer are covalently attached to one another, and the contact lens has a layered structural configuration. In an exemplary embodiment, the contact lens does not comprise a second silicone-containing layer. In an exemplary embodiment, the contact lens described does not comprise a third hydrophilic polymer-containing layer. In an exemplary embodiment, the contact lens does not comprise either a second silicon-containing layer or a third hydrophilic polymer-containing layer. In an exemplary embodiment, the contact lens comprises an anterior surface and a posterior surface wherein the anterior surface is the first hydrophilic polymer containing layer and the posterior surface is the second hydrophilic polymer-containing layer. In an exemplary embodiment, the contact lens described in this paragraph comprises an anterior surface and a posterior surface wherein the anterior surface is the first hydrophilic polymer containing layer and the posterior surface is the second hydrophilic polymer containing layer and the first and second hydrophilic polymer containing layer are substantially identical to each other. In other cases, the first hydrophilic polymer-containing layer has a composition, dimension, or other characteristic independent of the second hydrophilic polymer-containing layer.

In an exemplary embodiment, for any of the contact lenses of the invention, the first hydrophilic polymer-containing layer and the silicone-containing layer are covalently attached through a sulfonyl moiety. In an exemplary embodiment, for any of the contact lenses of the invention, the first hydrophilic polymer-containing layer and the silicone-containing layer are covalently attached through an alkylene sulfonyl moiety. In an exemplary embodiment, for any of the contact lenses of the invention, the first hydrophilic polymer-containing layer and the silicone-containing layer are covalently attached through a dialkylene sulfonyl moiety. In an exemplary embodiment, for any of the contact lenses of the invention, the first hydrophilic polymer-containing layer and the silicone-containing layer are covalently attached through an ethylene sulfonyl moiety. In an exemplary embodiment, for any of the contact lenses of the invention, the first hydrophilic polymer-containing layer and the silicone-containing layer are covalently attached through a diethylene sulfonyl moiety. In an exemplary embodiment, for any of the contact lenses of the invention, the first hydrophilic polymer-containing layer and the silicone-containing layer are covalently attached through a thioether moiety.

In an exemplary embodiment, for any of the contact lenses of the invention, the first hydrophilic polymer-containing layer and the silicone-containing layer are covalently attached through a sulfonyl moiety and a thioether moiety. In an exemplary embodiment, for any of the contact lenses of the invention, the first hydrophilic polymer-containing layer and the silicone-containing layer are covalently attached through an alkylene sulfonyl moiety and a thioether moiety. In an exemplary embodiment, for any of the contact lenses of the invention, the first hydrophilic polymer-containing layer and the silicone-containing layer are covalently attached through a dialkylene sulfonyl moiety and a thioether moiety. In an exemplary embodiment, for any of the contact lenses of the invention, the first hydrophilic polymer-containing layer and the silicon-containing layer are covalently attached through an ethylene sulfonyl moiety and a thioether moiety. In an exemplary embodiment, for any of the contact lenses of the invention, the first hydrophilic polymer-containing layer and the silicone-containing layer are covalently attached through a diethylene sulfonyl moiety and a thioether moiety.

In an exemplary embodiment, for any of the contact lenses of the invention, the second hydrophilic polymer-containing layer and the silicone-containing layer are covalently attached through a sulfonyl moiety. In an exemplary embodiment, for any of the contact lenses of the invention, the second hydrophilic polymer-containing layer and the silicone-containing layer are covalently attached through an alkylene sulfonyl moiety. In an exemplary embodiment, for any of the contact lenses of the invention, the second hydrophilic polymer-containing layer and the silicone-containing layer are covalently attached through a dialkylene sulfonyl moiety. In an exemplary embodiment, for any of the contact lenses of the invention, the second hydrophilic polymer-containing layer and the silicone-containing layer are covalently attached through an ethylene sulfonyl moiety. In an exemplary embodiment, for any of the contact lenses of the invention, the second hydrophilic polymer-containing layer and the silicone-containing layer are covalently attached through a diethylene sulfonyl moiety. In an exemplary embodiment, for any of the contact lenses of the invention, the second hydrophilic polymer-containing layer and the silicone-containing layer are covalently attached through a thioether moiety.

In an exemplary embodiment, for any of the contact lenses of the invention, the second hydrophilic polymer-containing layer and the silicone-containing layer are covalently attached through a sulfonyl moiety and a thioether moiety. In an exemplary embodiment, for any of the contact lenses of the invention, the second hydrophilic polymer-containing layer and the silicone-containing layer are covalently attached through an alkylene sulfonyl moiety and a thioether moiety. In an exemplary embodiment, for any of the contact lenses of the invention, the second hydrophilic polymer-containing layer and the silicone-containing layer are covalently attached through a dialkylene sulfonyl moiety and a thioether moiety. In an exemplary embodiment, for any of the contact lenses of the invention, the second hydrophilic polymer-containing layer and the silicone-containing layer are covalently attached through an ethylene sulfonyl moiety and a thioether moiety. In an exemplary embodiment, for any of the contact lenses of the invention, the second hydrophilic polymer-containing layer and the silicone-containing layer are covalently attached through a diethylene sulfonyl moiety and a thioether moiety.

In an exemplary embodiment, for any of the contact lenses of the invention, the first hydrophilic polymer-containing layer and the silicone-containing layer are covalently attached through an ester moiety. In an exemplary embodiment, for any of the contact lenses of the invention, the first hydrophilic polymer-containing layer and the silicone-containing layer are covalently attached through an alkylene ester moiety. In an exemplary embodiment, for any of the contact lenses of the invention, the first hydrophilic polymer-containing layer and the silicone-containing layer are covalently attached through an ethylene ester moiety. In an exemplary embodiment, for any of the contact lenses of the invention, the first hydrophilic polymer-containing layer and the silicone-containing layer are covalently attached through a thioether moiety.

In an exemplary embodiment, for any of the contact lenses of the invention, the first hydrophilic polymer-containing layer and the silicone-containing layer are covalently attached through an ester moiety and a thioether moiety. In an exemplary embodiment, for any of the contact lenses of the invention, the first hydrophilic polymer-containing layer and the silicone-containing layer are covalently attached through an alkylene ester moiety and a thioether moiety. In an exemplary embodiment, for any of the contact lenses of the invention, the first hydrophilic polymer-containing layer and the silicone-containing layer are covalently attached through an ethylene ester moiety and a thioether moiety.

In an exemplary embodiment, for any of the contact lenses of the invention, the second hydrophilic polymer-containing layer and the silicone-containing layer are covalently attached through an ester moiety and a thioether moiety. In an exemplary embodiment, for any of the contact lenses of the invention, the second hydrophilic polymer-containing layer and the silicone-containing layer are covalently attached through an alkylene ester moiety and a thioether moiety. In an exemplary embodiment, for any of the contact lenses of the invention, the second hydrophilic polymer-containing layer and the silicone-containing layer are covalently attached through an ethylene ester moiety and a thioether moiety.

### E. Additives To Hydrophilic Layer

Another aspect of the invention provides for methods of incorporating additives to the Hydrophilic Layer to improve its properties.

In addition to the hydrophilic polymer population, an additional component may be added to the layer, either embedded or attached to the surface, which serves to mimic the function of the anchoring mucin layer that is present on the corneal surface. For example MUC1, MUC4, and MUC16 are the primary membrane-associated ocular mucins. These mucins complex with soluble mucins present in the tear film; MUC5AC is secreted by conjunctival goblet cells and MUC7 is produced by lacrimal acinar cells. These soluble mucins complex with the membrane bound anchored mucins and thus form a stable, flexible layer over the surface. Mucins are highly glycosylated molecules and the high content of polysaccharide present on the normal corneal surface serves to maintain hydrophilicity, hydration, and service as an “adhesive” or “sticky” middle of the tear film to ensure tear film stability.

To mimic the function of the anchored mucin layer, the hydrophilic layer may contain glycosylated mucins or mucin analogs including peptide or peptoid sequences, or it may contain naturally occurring polysaccharides. Examples of polysaccharides include hyaluronic acid, dermatan sulfate, chondroitin sulfate, keratin sulfate, heparin sulfate, dextran, or unsulfated forms of polysaccharide chains. Polysaccharides may also include carragennans, alginates, chitosan, etc.

Mucin mimetic or polysaccharide components may be added to the hydrophilic layer through functionalization with a corresponding reactive group that reacts with the hydrophilic polymers. For example, the polysaccharides can be functionalized with vinyl sulfone and then added directly to the coating reaction. This results in a hybrid polymer/polysaccharide layer. The mucin mimetic layer may consist of a single molecule or combinations of multiple molecules.

Mucin mimetic/polysaccharide components may also be added in a second step in which functionalized polysaccharide/mucin mimics are added to the reaction mixture after the initial hydrophilic polymer layer has formed. For example, following the PEG-vinyl sulfone/PEG-thiol coating, the lens can be dipped in a thiol modified hyaluronic acid. The PEG surface contains an excess of vinyl sulfone groups and therefore the thiol modified hyaluronic acid reacts and yield a pendant hyaluronic acid layer. These processes could be repeated with any suitable ionic polymer species functionalized with corresponding functional groups.

Lenses functionalized with pendant polysaccharide groups enables complexation with the natural soluble mucins, glycosylated proteins, and soluble saccharides that are normally present in the tear film. This lens configuration, with a highly hydrated polymer layer combined with a mucin mimetic/polysaccharide layer has a unique ability to complex with natural tear film mucins and therefore dramatically improves comfort. The combination of the bulk crosslinked hydrophilic layer with the embedded or pendant polysaccharides confers additional benefit beyond the benefits of just the hydrophilic layer or just the mucin mimetic layer.

In a preferred embodiment, the lens complexes soluble mucins from the natural tear film in quantities higher than observed with standard contact lens materials. In a preferred embodiment, the lens of this invention results in a stabilized tear film, with increased tear film break-up times.

### F. Methods of Making a Coated Contact Lens or Multi-Layered Contact Lens

Another aspect of the invention provides for methods of making described coated and/or layered contact lenses.

In some embodiments, the method includes the steps of reacting a surface of a contact lens with a hydrophilic polymer solution. The hydrophilic polymer solution may contain one or more subpopulations or species that are adapted to react to form a coating on at least a portion of the contact lens. In some cases, the hydrophilic polymer solution reacts to form a cross-linked coating on the contact lens. The coating may be partially or substantially completely cross-linked.

As shown in FIG. 3A, the hydrophilic polymer solution may include a first polymer species with a reactive group A and a second polymer species with a reactive group N. The hydrophilic polymer layer may be formed on the contact lens by reacting the reactive groups on the first and second polymer species to form the cross-linked hydrophilic polymer layer. As shown in FIG. 3B, the reactive groups A and N may form a covalent linkage 54 between the first and second polymer species to thereby cross-link the two species and result in a hydrophilic polymer layer. In some cases, the reaction between the first and second reactive groups on respective polymer species forms a hydrogel.

As described, any suitable reaction may be employed to form the hydrophilic polymer layer. These include (without limitation) nucleophilic conjugate reactions, Michael-type reactions (e.g. 1,4 nucleophilic addition reactions), and/or click reactions. In some cases, the reactive groups A and N are an electron pair accepting moiety and a nucleophilic moiety respectively.

Additionally, in some variations, the polymer species or subpopulation with in the hydrophilic polymer layer may include PEG species. In some cases, a first PEG species reacts with a second PEG species to form the hydrophilic polymer layer. For example, the first PEG species may include an electron pair acceptor adapted to react to a nucleophilic reactive moiety of a second PEG species to covalently link the PEG species.

Some embodiments provide for a covalent attachment between the hydrophilic polymer layer and the lens core or layer. For example, one or more of the polymer subpopulation or species within the hydrophilic polymer layer or solution may be adapted to react to the lens core to form a covalent attachment between the hydrophilic layer and the lens core. In some cases, the method of hydrophilic polymer layer attachment includes the step of reacting at least one of the polymer species with reactive sites on the surface of the core to form covalent bonds between the polymer species and the core surface.

Referring again to FIGS. 5A-5C, a first polymer species P1 may include a reactive group A that is adapted to react to a reactive group N2 of the core 60 surface. The reaction between the A and N2 groups results in a covalent linkage 61 between the first polymer species P1 and the core 60. As shown, the reactive group A may also be adapted to react with another reactive moiety N1 of a second polymer species P2 to form the hydrophilic polymer layer. As such, a first reaction between P1 and P2 forms the hydrophilic polymer layer and a second reaction couples the hydrophilic polymer layer to the core.

In some cases, the same reactive group A on the first polymer species P1 is capable of reacting to either the reactive moiety N1 or N2. In one variation, a first portion of the reactive A groups react to the N1 moiety and a second portion of the reactive groups react to the N2 moiety. In some embodiments, the first and second portions of the reactive A groups are on the same molecule of a polymer species. In further variations, the first and second portions of the reactive A groups are on different branch arms of the same polymer species. The dual reactions between P1 and P2, and P1 and core may occur in the same reactive vessel and during the same reaction time (or overlapping in some portion of the reaction time).

As described, any suitable reaction may be employed to form the hydrophilic polymer layer and attach the hydrophilic polymer layer to the lens core. These include (without limitation) nucleophilic conjugate reactions, Michael-type reactions (e.g. 1,4 nucleophilic addition reactions), and/or click reactions. For example, the plurality of reactions may all be nucleophilic conjugate reactions. Alternatively, the plurality of reactions may be different types of reactions.

In some embodiments, the first and second reactions are nucleophilic conjugate reactions, more particularly, both are 1,4-nucleophilic addition Michael-type reactions. By way of example, in some embodiments, the nucleophilic reactive moiety of the first macromer population comprises a thiol group and the electron pair accepting moiety of the second macromer population comprises a sulfone group.

In other embodiments of the method the first and second nucleophilic conjugate reactions may be described more broadly as a “Click” type reaction. Click reactions, as originally described by Karl Sharpless and others, refer to modular assembly of macromolecules that are typified as occurring in an aqueous environment, delivering high yield as a result of being driven to completion by large thermodynamic force, and creating substantially no byproducts, or byproducts that are non-toxic to biological systems. The click reactions are advantageous for application toward the manufacture of contact lenses because the lenses may be reacted in an aqueous solution, without toxic byproducts, rapidly, and to high yield.

Other examples of click type reactions that could be used to attach branched polymers in our immersive dip coating process including (a) general thiol-ene click reactions in general, (b) [3+2] cycloadditions, including the Huisgen 1,2-dipolar cycloaddition, (c) Diels-Alder reaction, (d) [4+1] cycloadditions between isonitriles (isocyanides) and tetrazines, (e) nucloephilic substitution especially to small strained rings like epoxy and aziridine compounds, (f) carbonyl-chemistry-like formation of ureas, and (g) addition reactions to carbon-carbon double bonds, such as involve dihydroxylation or the alkynes in the thiolyne reaction.

In a particular embodiment, the method of making the described coated lens includes the steps of reacting an outer surface of the contact lens with a first PEG species of a hydrophilic polymer solution, wherein the first PEG species comprises an electron pair accepting moiety and a first portion of the electron pair accepting moiety forms a covalent attachment to the outer surface of the contact lens through a first nucleophilic conjugate reaction; and reacting the first PEG species of the hydrophilic polymer solution with a second PEG species of the hydrophilic polymer solution, the second PEG species comprising a nucleophilic reactive moiety adapted to covalently link to a second portion of the electron pair accepting moiety of the first PEG species in a second nucleophilic conjugate reaction to thereby at least partially cross-link the first and second PEG species, wherein a PEG hydrogel coating is formed and covalently attached to the outer surface of the contact lens by the first and second nucleophilic conjugate reactions.

In additionally embodiments, the method includes activating a surface of the lens core. Activating the surface may form a plurality of chemically reactive sites on the surface. The reactive sites may be, for example, nucleophilic sites for reaction with a hydrophilic polymer.

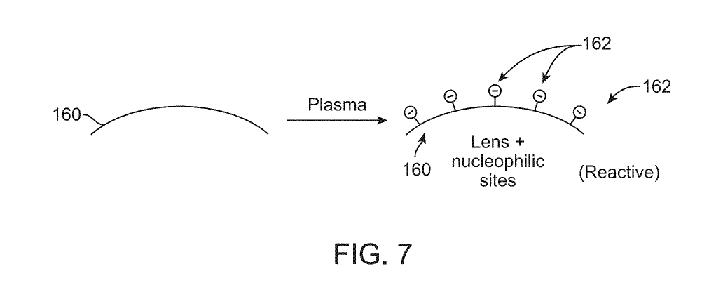

Referring to FIG. 7, a lens 160 without reactive sites is shown with a plurality of reactive sites 162 following an activation or modification process. In some cases, a plasma process is used to activate the surface of a core lens. The activation process may include the step of exposing the outer surface of the lens core to gas plasma. In some embodiments, the lens is transferred to a holding device, typically metal, and placed in a vacuum plasma chamber. The lens is plasma treated in an atmospheric plasma to form reactive sites on the surface. In some cases, an atmospheric plasma is applied to lens at 200 mTorr for about 3 minutes to thereby result in nucleophilic functional sites on the lens. In some embodiments, the lens are dehydrated prior to the plasma treatment.

In further variations, the contact lens surface may be activated through plasma treatment, preferably in oxygen or nitrogen gas. For example, the contemplated process may include activating a core material in a nitrogen plasma.

In other embodiments, activation of the contact lens surface can also occur through exposure to increasing pH's, for example solution pH of above 11.

In further embodiments, activation can also occur by modifying the monomer mix to include groups that are reactive to the branched hydrophilic coating polymers. Activation of the monomer mix can be a direct activation, or activation with a protected group that is cleaved, for example by light or changing pH. In other cases, plasma polymerization of functional silanes including mercapto and amino silanes may be used for activation. Additionally, plasma polymerization of allyl alcohol and allyl amine can also be used for activation.

In some embodiments, the core activation or modification step results in a reactive group N2 (shown in FIG. 5B) that is capable of reacting with at least one of the polymer species of the hydrophilic polymer layer. In some cases, at least one of the polymer species in the hydrophilic polymer layer reacts with a portion of the plurality of reactive sites on the core outer surface to form a covalent attachment between the hydrophilic polymer layer and the core surface. In some cases, the lens core is activated prior to the formation of the hydrophilic polymer layer on the core surface.

In some embodiments, the process of making the coated lens includes the step of reacting the activated core surface with a population of functionalized hydrophilic polymers. For example, the hydrophilic polymers may include a population of functionalized branched hydrophilic macromers with a first subpopulation functionalized with a nucleophilic reactive moiety and a second subpopulation functionalized with an electron pair accepting moiety. In further embodiments, the method may include reacting the functional moieties of two macromer subpopulations with each other in a first nucleophilic conjugate reaction to form covalent linkages between the two macromer subpopulations, thereby forming a cross-linked polymer network.

The method may also include reacting the electron pair accepting moieties of second macromer subpopulation and the nucleophilic moieties of the activated lens core surface in a second nucleophilic conjugate reaction to covalently attach the electron pair accepting moieties to the lens core surface. The first and second nucleophilic conjugate reactions, when complete, yield a contact lens that has a lens core with a cross-linked hydrophilic coating layer covalently attached thereto.

As described, the first and second nucleophilic conjugate reactions may be of the same type with the reactions differing by having different reactants. The two reactions may involve the same electron pair acceptor, such as the hydrophilic polymer species comprising an electron pair accepter that can participate in a plurality of reactions. The plurality of reactions may differ by having distinct nucleophilically-reactive parent molecules, in one case, a hydrophilic polymer species with a nucleophilic moiety, and in the second case, a silicone-based polymer of the lens core with a nucleophilic moiety.

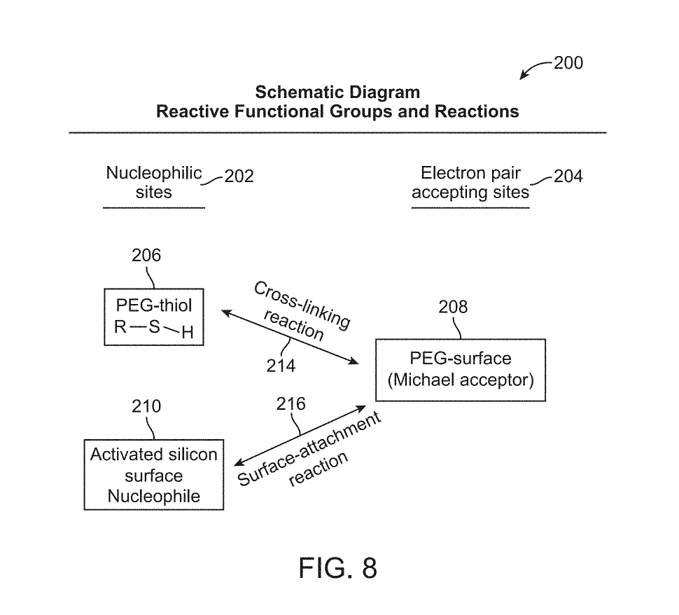

Referring to FIG. 8, a schematic diagram 200 of two exemplary conjugate addition reactions 214, 216 and the principal reactants are shown. The principal reactants can be understood as nucleophilic moieties 202 and electron pair accepting moieties 204. In a first reaction, a reactant having nucleophilic functional moiety, such as PEG-thiol 206, reacts with a reactant having an electron pair accepting functional moiety 204, such as PEG-sulfone 204; the product of the reaction 214 is a linked pair of PEG molecules, linked by way of a central thioether bond. As the reaction proceeds among the functionalized PEG molecules, the PEG takes the form of a linked network, and inasmuch as a PEG network is hydrophilic, in an aqueous environment, the network takes the form of an integrated hydrogel.

In a second reaction 216, a reactant 204 having an electron pair accepting functional moiety, such as PEG-sulfone 204, reacts with a nucleophilic site on the surface of the silicone-based lens core 210; the product of this second reaction 216 is a covalent bond between the PEG-sulfone and the lens core. As above, inasmuch as the individual molecular that covalently link to the activated silicone-based core also are included as a constituent of a hydrogel structure, the hydrogel structure, as a whole, becomes covalently linked lens core.

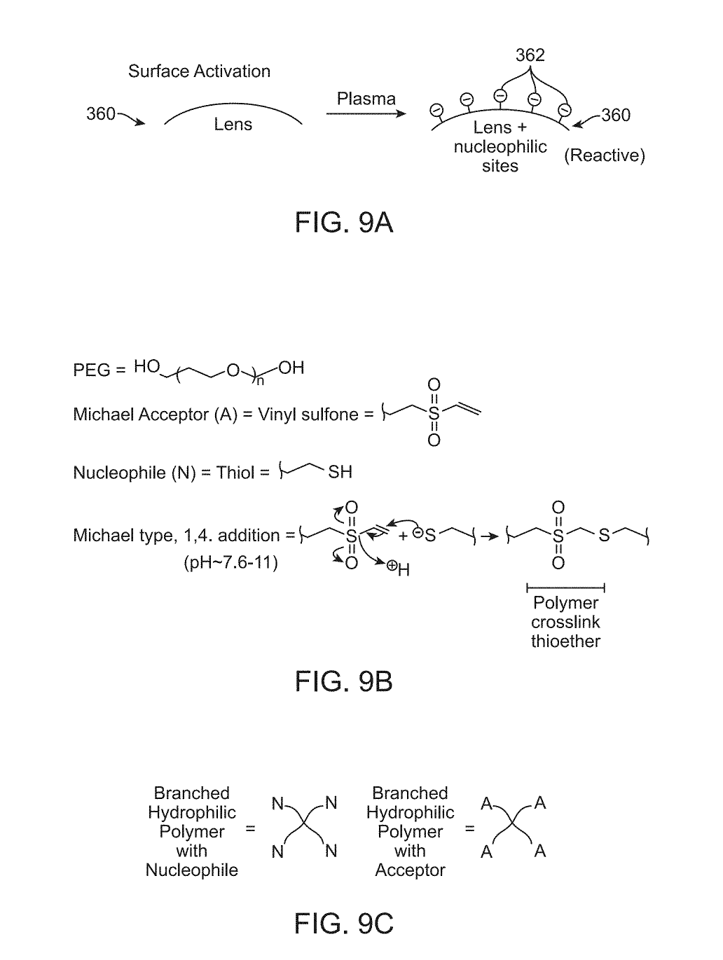

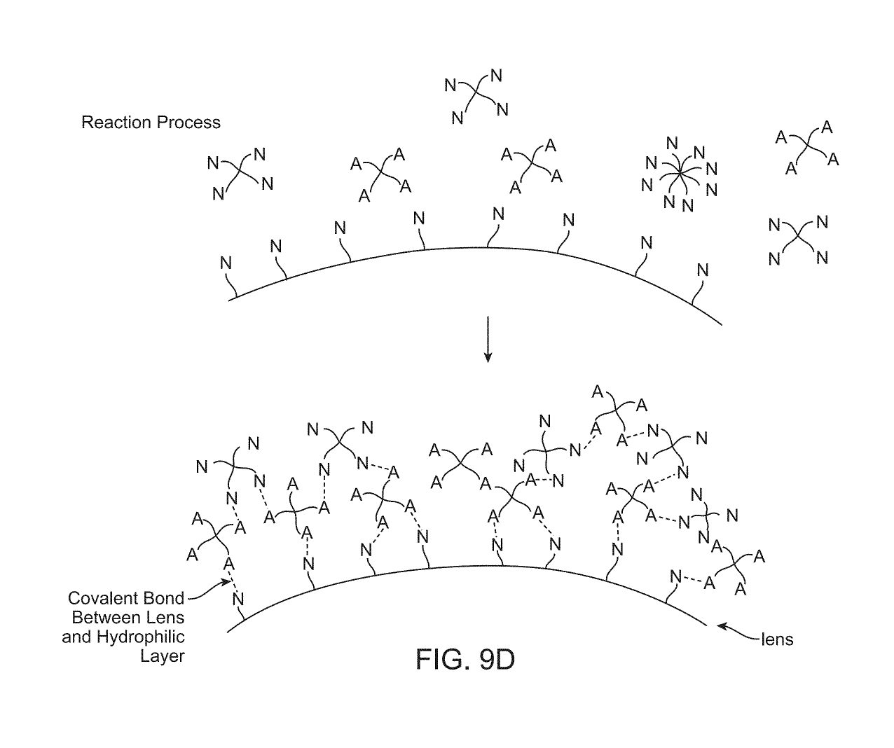

FIG. 9A-9D show more detailed and particular aspects of reactants and reactions, as depicted schematically in FIG. 8. FIG. 9A shows a silicone-based lens core being activated by a plasma treatment to yield a lens surface covered with a bed of activated nucleophilic sites. FIG. 9B shows the structure of examples of reactants, including a PEG molecule, a Michael-Type electron acceptor such as a vinyl sulfone moiety, a nucleophile functional group such as a thiol, and the detail of the Michael type reaction itself.

FIGS. 9C-9D show a reaction process whereby two subpopulations of branched hydrophilic polymer species, a first subpopulation with a nucleophile functionality (N) and a second subpopulation with an electron pair accepting functionality (A) are in a reaction solution that bathes a nucleophilically activated (N) lens core. In the lower portion of FIG. 9D, per the first reaction as depicted in FIG. 8, reaction individual members of the two subpopulations have begun to link together by way of their functional groups, to form a hydrogel network. And, per the second reaction as depicted in FIG. 8, electron pair accepting moieties (A) of hydrophilic polymers engage in covalent linking with the nucleophilic sites on the lens surface, thereby covalently attaching the hydrogel network to the lens surface.

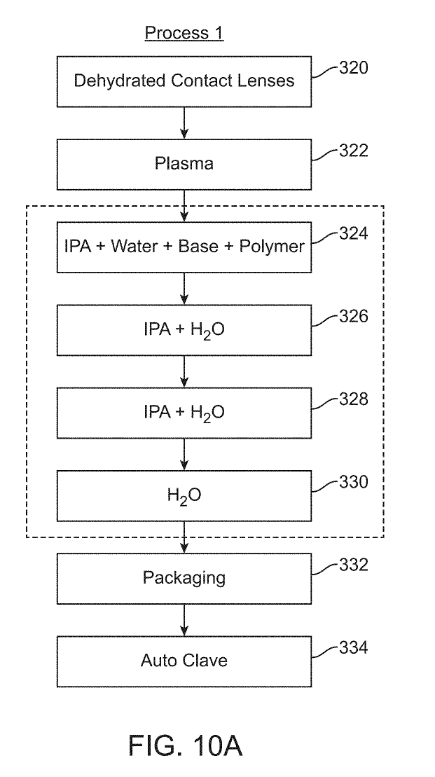

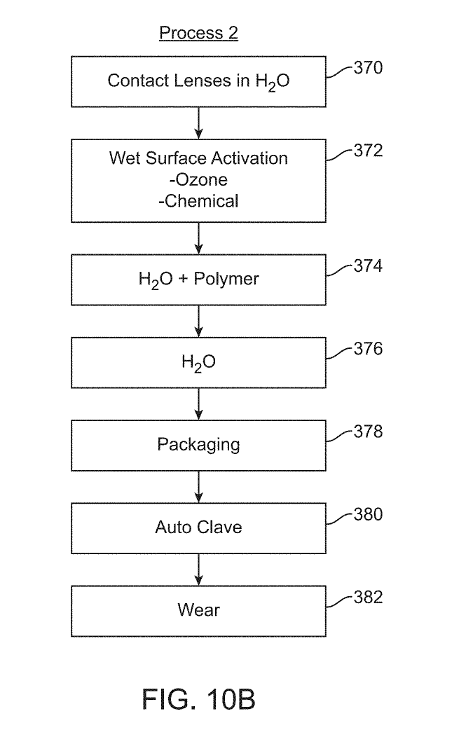

FIGS. 10A-10B provide flow diagrams of two variations of processes for making a contact lens with a covalently attached hydrogel membrane. FIG. 10A shows a process that includes a plasma activation method. Such plasma treatment may include exposure to any of an oxygen plasma or a nitrogen plasma. FIG. 10B shows a process that includes a chemical or “wet” activation method.

As described in FIG. 10A, a contact lens 320 plasma treated 324 to form a plurality of reactive sites on the contact lens. This may be accomplished by placing the lens into a vacuum plasma chamber. In some embodiments, the lens is transferred to a holding device, typically metal, and placed in a vacuum plasma chamber. The lenses are plasma treated in an atmospheric plasma at 200 mTorr for about 3 minutes, thereby creating nucleophilic functional sites on the lens. As described, the lens may be in a dehydrated state prior to the plasma treatment.

Referring still to FIG. 10A, the activated lens core is placed into a solution that includes coating polymer and/or coating polymer species or precursors 324. The coating polymer may be any of the described hydrophilic polymers described including a hydrophilic polymer population including subpopulations of functionalized branched PEG species. In some cases, the solution also includes isopropyl alcohol and water. The solution may have a pH>7. The solution may be agitated to create a well-stirred bath and the lenses incubate in the solution for some period of time. In some cases, the incubation time is about 50 minutes.

Optionally, the coating process may include extraction steps to remove an unwanted component from the coated lens. For example, where a silicone-based lens core is used for a base or substrate, unreacted silicone molecules in the lens cores are extracted or diffused out of the lenses. Advantageously, the extraction process removes raw lens core material (e.g. raw silicone for a silicone-containing core) that may leach out of the lens into the ocular region. As such, further steps of the process may include transferring the lens to a solution of isopropyl alcohol and water for a period of time such as about 50 minutes 326 to continue extracting unreacted silicone molecules from the lens cores. Additionally, as a second rinse 328, the lens may be transferred to a fresh solution of isopropyl alcohol and water for a period of time such as about 50 minutes to further extract unreacted silicone molecules from the lens cores. In some variations, the lens may also be transferred into a water bath 330 to equilibrate in water for a period of time (e.g. about 50 minutes).

Additionally, as shown in FIG. 10A, the lens may be transferred to a packaging container with a packaging solution 332. The lens may also be autoclaved 334. In some cases, the lens is autoclaved at about 250° F. for about 30 minutes.

FIG. 10B describes a wet-activation process for activating a lens core and coating the activated core. The process may begin with a lens in a hydrated state 370. The next step may include activating the hydrated surface lens core 372. This may be accomplished by a plasma or chemical treatment. For example, ozone may be used to activate the core surface. Once activated, the activated lens may be placed into a solution containing the coating material 374. The solution may include a hydrophilic polymer solution as described and water. In some cases, the solution is at a pH>7. The solution may be agitated to create a well-stirred bath and the lens incubates therein. In some cases, the lens incubates for about 50 minutes.

Next, the lens may be transferred to a water bath to equilibrate in water 376. The equilibration step may also serve to wash excess polymer from the lens. The lens may be equilibrated in water for about 50 minutes. The lens may be transferred to a packaging container with packaging solution 378. Additionally, as another step, the lens may be autoclaved. In some cases, the lens is autoclaved at about 250° F. for about 30 minutes. After the autoclave step, the resulting coated lens is ready for use 382.

Advantageously, the methods described herein provide for a cost-effective coating process that can be integrated with contact lens manufacturing processes currently employed in the industry.

Some embodiments of the method may be understood as an immersive method, wherein activated lens cores are immersed in a reaction solution within a stirred vessel, the solution including hydrophilic macromer reactants, and the reaction vessel operated to achieve appropriate reaction conditions. The reaction vessel and aspects of the conditions, in biochemical engineering terms, may be understood as occurring in a continuously stirred reaction tank (CSTR). In typical embodiments, the reacting steps occur within a reaction solution that has an aqueous solvent. Such the aqueous solvent may include any one or more of water, methanol, ethanol, or any suitable aqueous solvent that solubilizes PEG.

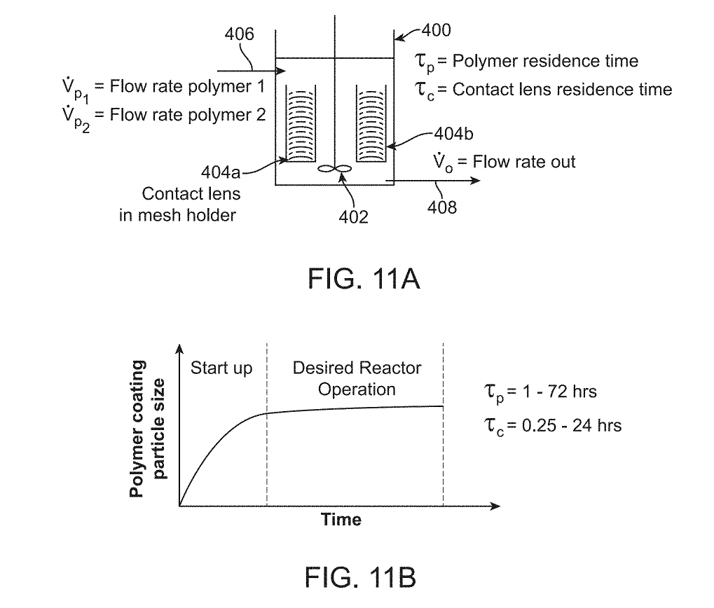

FIG. 11A provides a schematic view of a continuously stirred tank reactor (CSTR) 400 suitable for performing the reaction described. The CSTR 400 includes an agitator 402 for stirring the reaction contents within the tank. A feeding line or conduit 404 allows input or inflow 406 of reaction solutions including a hydrophilic polymer solution containing at least one polymer species. As shown, first and second polymer species flow into the CSTR 400. In some cases, the first and second polymer species have different flow rates VP1 and VP2 respectively. In other cases, the flow rates may be the same.

FIG. 11A shows a plurality of contact lenses 404a and 404b in the CSTR 400. In some cases, the contact lenses may be held in a mesh holder with openings or sufficient porosity to allow contact between the held lenses and the solution in the CSTR.

FIG. 11A also shows an output or outflow opening or conduit 408 for removing fluid from the CSTR 400. In some cases, the removed fluid is spent reaction fluid. The flow rate of the removed fluid may be designed as V0.

In some cases, Tp indicates the polymer residence time and TC indicates the contact residence time in the CSTR 400. FIG. 11B shows the relationship between polymer coating particle size as a function of time in a CSTR 400 where TP is 1-72 hours and TC is 0.25-24 hours.

In some variations, within the reaction solution, the total hydrophilic macromer concentration in the solution typically ranges between about 0.01 (w/v) % and about 0.50 (w/v) %. In some embodiments, the first and second macromer subpopulations are present in the solution at substantially equivalent concentrations. However, in other embodiments, the concentration of the reactive moiety of the second macromer subpopulation (an electron pair accepter) exceeds the concentration of the reactive moiety of first macromer subpopulation (a nucleophile).

Having an excess of electron pair reactive moieties with respect to the nucleophilic reactive moieties can be advantageous for the reactions included herein for the purpose of forming embodiments of hydrogel-coated contact lenses in that the electron pair accepting moieties of the hydrophilic polymer subpopulation so-functionalized can participate in two reactions. The polymer subpopulation functionalized with the electron pair acceptors participates (1) in covalent cross linking with the subpopulation functionalized with nucleophiles and (2) covalent attachment to nucleophilic sites on the silicone-based core lens surface. In contrast, the polymer subpopulation functionalized with a nucleophilic moiety engages only in the single reaction wherein it engages the polymer subpopulation functionalized with the electron pair accepting moiety.

The reactant concentration may also be appropriately expressed in terms of the relative concentrations of the reactive moieties of the participant macromers, rather than the concentrations of the macromers themselves. This follows from the possible variations in the degree to which the macromers are decorated with the function moieties that actually participate in the reactions. Accordingly, in some reaction embodiments, the concentration of the reactive moiety of the second macromer subpopulation exceeds the concentration of the reactive moiety of the first macromer subpopulation by at least about 1%. In more particular embodiments, the concentration of the reactive moiety of the second macromer subpopulation exceeds the concentration of the reactive moiety of the first macromer subpopulation by an amount that ranges between about 1% and about 30%. And in still more particular embodiments, the concentration of the reactive moiety of the second macromer subpopulation exceeds the concentration of the reactive moiety of the first macromer subpopulation by an amount that ranges between about 5% and about 20%.

Returning now to aspects of the reaction conditions, in some embodiments, the reacting steps are performed for a duration of between about 5 minutes and about 24 hours. In particular embodiments, the reacting steps are performed for a duration of between about 0.5 hour and about 2 hrs. In some embodiments, the reacting steps are performed at a temperature at a range between about 15° C. and about 100° C. In more particular embodiments, the reacting steps are performed at a temperature at a range between about 20° C. and about 40° C. In some embodiments, the reacting steps are performed at a pH between about 7 and about 11.

In some embodiments, the activated lens material is incubated in a dilute reaction solution containing 4-arm branched, 10 kDa PEG end functionalized with thiol groups, and 8-arm branched, 10 kDa PEG end functionalized with vinyl sulfone groups. The dilute solution contains between 0.01 and 0.5% total polymer, with a 10% excess of vinyl sulfone groups. The reaction can be performed in aqueous conditions, methanol, ethanol, or other solvents in which PEG is soluble. The reaction can be performed at a range of temperatures between about 15 degrees C. and about 100 degrees C. The reaction can be performed from between about 5 minutes and about 24 hours. The reaction can be performed at basic pH's, preferably in the range of 7-11.

As polymer reaction proceeds in the dilute solution, hydrogels (e.g. cross-linked hydrophilic polymer particles) are formed as branched polymers react with each other. Reaction progress can be monitored using dynamic light scattering techniques to measure hydrogel particle size and/or macromer aggregation level as the hydrogel network is forming. Temperature, pH, convection speed, and concentration will influence reaction rate and hydrogel particle size and formation rate. Hydrogel particles that are smaller than visible light will not cause optical distortions in the contact lens. Layer thickness can be regulated by monitoring hydrogel formation during the course of reaction.

In some variations, polyethylene glycol is the hydrophilic polymer. However, other multifunctional natural and synthetic hydrophilic polymers can also be used, for example poly(vinyl alcohol), poly(vinylpyrrolidinone), Poly(N-isopropylacrylamide) (PNIPAM) and Polyacrylamide (PAM), Poly(2-oxazoline) and Polyethylenimine (PEI), Poly(acrylic acid), Polymethacrylate and Other Acrylic Polymers, Polyelectrolytes, hyaluronic acid, chitosan, dextran.

In other embodiments, the methods include the step of forming a cross-linked hydrophilic polymer layer on a lens surface that is covalently attached to the contact lens. Covalent linkages between the branched hydrophilic polymers may occur due to Michael type nucleophilic conjugate addition reaction between vinyl sulfone and thiol and covalent linkages between the hydrophilic polymer and the lens surface occur due to conjugate addition reaction between vinyl sulfone and nucleophiles generated during the activation step. In some cases, reactivity of nucleophiles will increase with rising pH as molecules are increasingly deprotonated.

In further variations, any general Michael type reaction between enolates and conjugated carbonyls can also be used. For example, acrylate, methacrylate, or maleimide can be substituted for vinyl sulfone. Other examples include the Gilman reagent as an effective nucleophile for addition to conjugated carbonyls. The stork enamine reaction can be performed using enamines and conjugated carbonyls.

Additional covalent reaction mechanisms include hydroxylamine reaction with electrophiles such as aldehyde or ketone to produce oxime linkages.

Additional covalent reaction mechanisms include reaction of N-Hydroxysuccinimidyl esters with amines.

Additional covalent reaction mechanisms include isocyanates reaction with nucleophiles including alcohols and amines to form urethane linkages.

In another embodiment, a PEG containing layer can be attached to a silicone containing lens layer using cast molding techniques. First, the silicone containing layer is modified to ensure surface groups are present that will react covalently with the PEG macromers. Second, molds are prepared that contain a top part and a bottom part in the same or similar shape as the silicone containing layer. The silicone containing layer is placed into the mold along with the liquid macromer PEG solution and the mold halves are placed together. The PEG can cure thermally for approximately 1 hour and the mold is taken apart.

The PEG containing layer can also be attached to the silicone containing layer using a dip coating method. First, the silicone containing layer is modified to ensure surface groups are present that will react covalently with the PEG macromers. For example, surface groups can be generated in a plasma treatment step, or by incubating in a basic solution, or by including reactive groups in the monomer mix. Next, a dip coating solution is prepared that consists of a dilute solution of reactive, branched, hydrophilic polymers. The activated lens is placed in the dip coating solution and incubated for 1-24 hours. Following incubation, the lens is rinsed thoroughly and then autoclaved in an excess volume of buffer solution prior to measuring captive bubble contact angles.

In alternative method, the hydrophilic polymer layer can be covalently attached to the silicone containing layer using another dip coating method. First, the silicone containing layer can be modified to create surface chemical moieties that are covalently reactive to the hydrophilic macromers. For example, surface groups can be generated in a plasma treatment step, or by incubating in a basic solution, or by including reactive groups in the monomer mix. Next, a dip coating solution can be prepared that consists of a dilute solution of reactive, branched, hydrophilic polymers. For example, the dilute solution can consist of a branched poly(ethylene glycol) end functionalized with vinyl sulfone and thiol in a solution containing 0.2 M triethanolamine. The activated lens is placed in the dip coating solution and incubated for 1-24 hours at a temperature between about 20° C. and about 60° C. Following incubation, the lens is rinsed thoroughly and then autoclaved in an excess volume of phosphate buffered saline.

In another embodiment, the invention provides a method of making a contact lens described herein. The method comprises contacting an activated lens and a dip coating solution, thereby making a contact lens. In another embodiment, the method further comprises activating a lens, thereby creating an activated lens. A lens can be activated through a method known to one of skill in the art or a method described herein, such as plasma treatment or incubation in a basic solution, or by including reactive groups in the monomer mix. In an exemplary embodiment, the contacting takes place for between 1-24 hours, or from 1-12 hours, or from 12-24 hours, or from 6-18 hours. In an exemplary embodiment, the method further comprises rising the lens after the contacting step. In an exemplary embodiment, the method further comprises autoclaving the lens after the contacting step. In an exemplary embodiment, the method further comprises autoclaving the lens after the rinsing step.

In an exemplary embodiment, the invention provides a method of making a contact lens described herein. A lens can be activated by including reactive groups in the monomer mix. In an exemplary embodiment, the activated contact lens is placed in a solution containing the functionalized coating components. The activated contact lens in the coating solution is then placed in an autoclave at 250 degrees Fahrenheit during which the polymer coating covalently binds to the activated lens surface and becomes simultaneously sterilized.

In another embodiment, an alternative method of forming a contact lens includes a spray coating approach wherein a reactive ultrasonic spray coating is used to coat substrates with a thin, adhered layer of cross-linked hydrogel. A two-component hydrogel, comprising branched PEG end-capped with vinyl sulfone, and branched PEG end-capped with thiol, was used to produce the cross-linked thin films. The two components are simultaneously dripped onto an ultrasonic spray nozzle where they are combined and atomized into small droplets, which then are accelerated to the substrate in an air sheath. The rate of reaction is adjusted to ensure that reaction is fast enough that a solid structure forms on the surface, but slow enough that the components do not instantly polymerize upon mixing at the nozzle.

An alternative spray method, considered appropriate for scaled manufacturing, is ultrasonic spray coating, a technique that enables precise, thin film coatings. It has been employed previously for stents and in the microelectronics industry, and is currently used in several high volume manufacturing lines. A state of the art Sonotek instrument was used to form coated contact lens prototypes. This technology enables 3D printing, thus potentially providing a platform for constructing complicated lens structures with integrated sensors or electronics.

The Sonotek instrument has an ultrasonically driven spray nozzle with two feed lines that deposit solution onto the tip. A two-component hydrogel system involves dissolving the PEG vinyl sulfone component in methanol containing triethanolamine (TEOA; acting as an organic base) and the PEG thiol component in pure methanol. The two solutions are delivered to the nozzle tip at a rate of 5 microliters per minute and the concentration of each PEG component is adjusted such that equal volumes of each component mix to achieve a 10% molar excess of vinyl sulfone groups. When the solutions are deposited on the ultrasonic tip, they mix and are atomized into droplets that are approximately 20 microns in diameter. A pressured air sheath then accelerates the droplets onto the surface to be coated. By including FITC-malelimide in the PEG vinyl sulfone component, mixing and crosslinking that result in film deposition can be films. A concentration of TEOA and identified that at a molar ratio of TEOA:SH of 6:1 could deposit a uniform crosslinked hydrogel on a variety of substrates, including pure silicone and silicone hydrogel core lenses. An alternative aqueous spray coating method was also tested and was shown to be feasible, however for the contact lens substrates, the methanol process advantageously produces a highly uniform film of ˜5 microns. The contact angle measurements on coated lenses demonstrated the integrity of the deposited film.

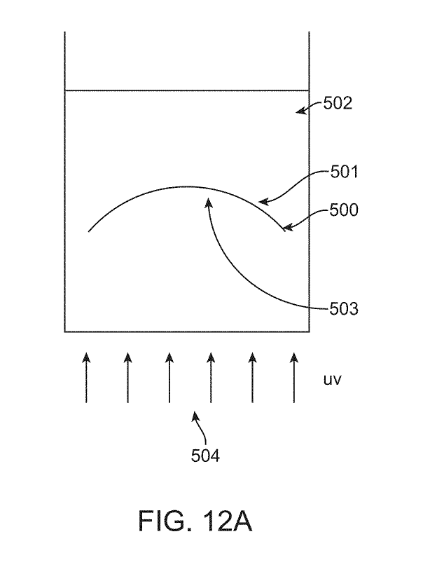

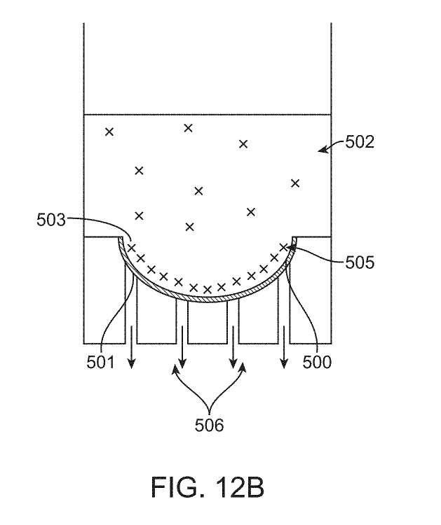

FIGS. 12A and 12B depict alternative embodiments of methods of the technology that are directed toward making lenses with a covalently attached bilateral hydrophilic coating layer, in which the hydrophilic coating layer sides differ in composition or depth. In some instances, it may be advantageous to produce a contact lens that is asymmetric (convex side vs. concave side) with regard to the thickness or composition of the hydrogel coating that is associated with the two surfaces, respectively. For example, it may be advantageous to form a hydrophilic coating layer on the concave (or posterior) lens surface that is thicker than the layer on the convex (or anterior) lens surface, in order to hold a greater volume of aqueous tears against the cornea and prevent symptoms of dryness.

FIG. 12A shows a method to produce a lens with a thicker hydrophilic layer on the concave surface 503 in which a lens core 500 containing a UV blocking agent is dipped into a non-mixed solution 502 of coating polymer, and then exposed to UV light 504. UV light accelerates the reaction between polymers as well as the reaction between polymer and surface. The light strikes the lens on a vector that is perpendicular to the lens surface, directly onto the concave side 503 and through the convex side 501. Due to the UV blocking agent present in the lens, the concave side 503 is exposed to a higher dose of UV light, while the convex side 501 receives a relatively lower dose. This asymmetric UV dosing creates layers of varying thickness. To achieve complete independent variation in layer thickness control, light dosage of varying intensity can also be used to shine from each side.

FIG. 12B shows an alternative method for producing a thicker hydrophilic coating layer on the concave surface 503 of the lens 500. As shown, the convex surface 501 of the lens 500 is held in a vacuum chuck 506 while exposing the concave surface 503 to the coating polymer 502. The vacuum suction pulls the aqueous solvent through the lens 500 while concentrating coating polymer at the lens interface at the concave surface 503. After achieving a desired layer thickness, the lens 500 is removed from the chuck 506. In some variations, the lens 500 is then placed into a well-mixed bath of coating polymer, to continue building the hydrophilic coating layer on both sides of the lens.

### EXAMPLES

Additional properties of the highly oxygen permeable, hydrophilic, soft contact lens and the processes for forming fabricating are illustrated in the Examples. The Examples are not intended to define or limit the scope of the invention.

### Example 1

Silicone Elastomer 14 mm disks with activator were made by combining polydimethylsiloxane (Gelest, Inc), methacryloxypropyltris silane (Gelest, Inc), glycidyl methacrylate (Sigma) at 5% concentration, and darocure then curing with ultraviolet light between glass slides for 5 minutes. The glass slides were separated and a 14mm punch was used to create the disks. The disks were then solvent extracted in 50% isopropyl alcohol for 30 minutes then washed 3 times in deionized water. The disks were then placed in a 10 ml vial where 2 ml of saline, and 20 ul of coating solution were added (10 ul of vinyl sulfone functionalized polyacrylamide and 20 ul of thiol functionalized polyethylene glycol). The vial was vortexed for 10 seconds, capped and placed in an autoclave at 250 degrees Fahrenheit for 30 minutes (standard contact lens sterilization protocol). Two sets of control lenses were made; one without activator and with coating solution; the second with activator and no coating solution. Following the autoclave cycle, all lenses were washed in water 4 times for 30 minutes each to remove all unreacted polymer from the solution and then tested for contact angle, lubricity, and water breakup time. Increased wettability, lubricity, and water break-up are observed due to phase separation of the polyethylene glycol component in the autoclave.

CONTACT ANGLE RESULTS:

| | Advancing | | Water |
|---|---|---|---|
| | Contact | | Breakup |
| | Angle | Lubricity | Time |
| Lens | (degrees) | (1-5 scale) | (1-5 scale) |
| Control (−) Activator | 90-110 | 1 | 1 |
| Control (+) Activator | 90-110 | 1 | 1 |
| (−)Coating | | | |
| Test lens (+) Activator, | 45-55 | 4.5-5 | 4 |
| (+) Coating | | | |

### Example 2

Silicone Hydrogel 14 mm disks were made by combining dimethacrylate polydimethylsiloxane (Gelest, Inc), methacryloxypropyltris silane (Gelest, Inc), dimethyl methacrate (Sigma), and darocure. Lenses were also made with chemical activator only, physical activator only, and a combination of both. The chemical activator used was a polyethylene glycol bifunctional linker of molecular weight 350 with a methacrylate group at one end and an amine salt on the other end used at a weight concentration of 0.2% w/v. The physical activator was a methacrylic acid used at a concentration of 1% w/v. The disks were then cured with ultraviolet light between glass slides for 5 minutes. The glass slides were separated and a 14 mm punch was used to create the disks. The disks were then solvent extracted in 50% isopropyl alcohol for 30 minutes then washed 4 times in deionized water. The disks were then placed in a 10 ml vial with 2 mL of 0.2 M TEOA, and 20 ul of coating solution were added (amine functionalized polyacrylamide and vinyl sulfone functionalized branched polyethylene glycol). The vial was vortexed for 10 seconds, capped and placed at 60 degrees Celsius for 90 minutes. Four sets of lenses were made; one without activator, one with chemical activator only, one with physical activator, and one with both chemical and physical activator. Following the coating processes, all lenses were washed in saline 4 times for 30 minutes each to remove all unreacted polymer from the solution and then tested for contact angle, lubricity, and water breakup time.

| | Advancing | Water | |
|---|---|---|---|
| | Contact | Break-up | |
| | Angle | Time | Manual |
| Activator | (degrees) | (s) | Lubricity |
| None | 95 | 0 | 0 |
| Amine | 53 | 0 | 1 |
| Carboxylic Acid | 50 | 0 | 1 |
| Combination | 40 | 25 | 6 |

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims. The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of coating a contact lens core comprising:
a. Reacting an outer surface of the contact lens core with a first polymer species of a hydrophilic polymer solution, wherein the lens core is about 75% to about 100% silicone, wherein the first polymer species comprises an electron pair accepting moiety and a first portion of the electron pair accepting moiety forms a covalent attachment to the outer surface of the contact lens through a first nucleophilic conjugate reaction; and
b. Reacting the first polymer species of the hydrophilic polymer solution with a second polymer species of the hydrophilic polymer solution, the second polymer species comprising a nucleophilic reactive moiety adapted to covalently link to a second portion of the electron pair accepting moiety of the first polymer species in a second nucleophilic conjugate reaction to thereby at least partially cross-link the first and second polymer species, wherein a polymer hydrogel coating is formed and covalently attached to the outer surface of the contact lens core by the first and second nucleophilic conjugate reactions.

2. The method of claim 1, further comprising modifying an outer surface of the lens core to form a plurality of chemically reactive nucleophilic sites on the outer surface.

3. The method of claim 1, further comprising modifying an outer surface of the lens core to form a plurality of moieties that physically attract the polymer species to the lens surface.

4. The method of claim 1, further comprising modifying an outer surface of the lens core to form a combination of a plurality of chemically reactive sites as well as a plurality of physically attractive sites on the outer surface.

5. The method of claim 1, further comprising exposing the outer surface of the contact lens to a gas plasma treatment.

6. The method of claim 2, wherein the reactive nucleophilic sites on the outer surface include amines.

7. The method of claim 3, wherein the moieties on the outer surface include carboxylic acids.

8. The method of claim 1, further comprising modifying an outer surface of the lens core, wherein modifying includes the addition of an activator to a chemical mix used to form the lens core.

9. The method of claim 8, wherein the activator participates in a radical polymerization process of the chemical mix during fabrication of the lens core.

10. The method of claim 8, wherein the activator is a bifunctional polyethylene glycol.

11. The method of claim 10, wherein at least one moiety of the bifunctional activator does not participate in the radical polymerization process of the core lens during fabrication.

12. The method of claim 8, wherein the activator covalently bonds to a silane backbone of the lens core.

13. The method of claim 8, wherein the activator is N-(3-Aminopropyl)methacrylamide hydrochloride.

14. The method of claim 1, wherein reacting an outer surface of the contact lens with the first polymer species comprises reacting at least a portion of the plurality of reactive nucleophilic sites on the outer surface with a first portion of the electron pair accepting moiety on the first polymer species.

15. The method of claim 1, wherein the nucleophilic conjugate reactions are 1,4-nucleophilic addition reactions.

16. The method of claim 1, wherein the nucleophilic conjugate reactions are Michael-type reactions.

17. The method of claim 1, wherein the nucleophilic conjugate reactions are click reactions.

18. The method of claim 1, wherein the nucleophilic reactive moiety of the second polymer species is a thiol group and the electron pair accepting moiety of the first polymer species is a sulfonyl group.

19. The method of claim 1, wherein the first polymer species and the second polymer species are cross-linked through an aminoether moiety.

20. The method of claim 1, wherein the nucleophilic reactive moiety of the second polymer species is an amine group and the electron pair accepting moiety of the first polymer species is a sulfonyl group.

21. The method of claim 1, wherein the first polymer species and the second polymer species are cross-linked through an aminoether moiety.

22. The method of claim 1, wherein the nucleophilic reactive moiety of the second polymer species is an amine group and the electron pair accepting moiety of the polysaccharide species is a sulfonyl group.

23. The method of claim 1, wherein the first polymer species and the polysaccharide species are cross-linked through an aminoether moiety.

24. The method of claim 1, wherein the hydrophilic polymer solution comprises substantially equivalent concentrations of the reactive moieties of the first polymer species and second polymer species.

25. The method of claim 1, wherein the concentrations of the reactive moieties of the first polymer species exceeds the concentration of the nucleophilic reactive moiety of the second polymer species by about 1% to about 50%.

26. The method of claim 1, wherein the reacting steps are performed at a temperature between about 15 degrees Celsius and about 60 degrees Celsius.

27. The method of claim 1, wherein the reacting steps are performed at a temperature of 120 degrees Celsius and 17 barr pressure

28. The method of claim 1, wherein the reacting steps are performed at a pH between about 7 and about 12.

29. The method of claim 1, wherein the polymer hydrogel coating is substantially optically clear.

30. The method of claim 1, wherein the contact lens comprises a core consisting of silicone.
